(19) United States
(12) Patent Application Publication
(10) Pub. No.: US 2024/0366966 A1
(43) Pub. Date: Nov. 7, 2024

(54) DEVICE AND METHOD FOR REDUCING INJURY RESPONSE IN INJURED TISSUE

(71) Applicant: Actuated Medical, Inc., Bellefonte, PA (US)

(72) Inventors: Roger B. Bagwell, Bellefonte, PA (US); Maureen L. Mulvihill, Bellefonte, PA (US); Kyle W. Gheres, Pennsylvania Furnace, PA (US); Jenna K. Greaser, State College, PA (US); Kevin A. Snook, State College, PA (US)

(73) Assignee: Actuated Medical, Inc., Bellefonte, PA (US)

(21) Appl. No.: 18/743,214

(22) Filed: Jun. 14, 2024

Related U.S. Application Data
(63) Continuation-in-part of application No. 17/837,766, filed on Jun. 10, 2022.
(60) Provisional application No. 63/519,316, filed on Aug. 14, 2023.
(60) Provisional application No. 63/591,775, filed on Oct. 20, 2023.
(60) Provisional application No. 63/231,410, filed on Aug. 10, 2021.

(51) Int. Cl. A61N 7/00

(52) U.S. Cl. CPC A61N 7/00

(57) ABSTRACT

An assembly for reducing foreign body response in a subject caused by an invasive or non-invasive tissue injury at a target site. A base with aperture is secured to the subject in proximity to the target site with an implant extending therethrough. The base includes a sloped channel that receives an alignment tab of a housing, permitting vertical adjustment of the housing relative to the base along a continuum by rotation of the housing. The housing contains a transducer generating vibrations when activated, an acoustic horn in contact with the transducer transmitting the vibrations through the base aperture to the subject tissue at the target site, and a compressible polymer surrounding a portion of the horn at the terminal end of the housing that conforms to the subject's interface and transmits vibrations to the target site for reducing foreign body response or microgliosis in the subject.

### CLAIM OF PRIORITY

The present application claims the benefit of both U.S. Provisional Application Ser. No. 63/519,316, filed on Aug. 14, 2023, and U.S. Provisional Application Ser. No. 63/591,775, filed on Oct. 20, 2023, and is a continuation-in-part of co-pending U.S. application Ser. No. 17/837,766, filed on Jun. 10, 2022, which claims the benefit of U.S. Provisional Application Ser. No. 63/231,410, filed Aug. 10, 2021, the contents of all of which are incorporated herein by reference in their entireties.

### STATEMENT REGARDING FEDERALLY SPONSORED RESEARCH OR DEVELOPMENT

This invention was made with government support under EB028055 and MH131514 awarded by the National Institutes of Health. The government has certain rights in the invention.

### FIELD OF THE INVENTION

This invention relates to a device for delivering acoustic stimulation to injured tissue, including injury surrounding an implant, reducing bodily response to the injury.

### BACKGROUND

Implants, such as chronically implanted microelectrode arrays designed to interface with neural tissue, hold great potential for revolutionizing treatment of a range of medical conditions. Applications of neural implants include neural-based control of prosthetic limbs by amputees, brain-machine interfacing for paraplegics, selective ablation and/or inactivation of problematic neural pathways, or control or enhancement of organ function, to name a few. Programs like SPARC, the BRAIN Initiative, and BrainGate are bringing new neuroprosthetic devices to patients, and researchers predict that neural implants will be more widely implemented in humans in the next 10 years. Non-penetrating neural implant electrode arrays such as EEG electrodes and nerve cuffs have seen increased clinical application in recent years, but such systems have limited spatial resolution, making them less ideal for future applications requiring more precise stimulation or recording. Penetrating neural electrode arrays offer significantly improved temporal and spatial resolution but suffer from multiple complications which restrict their clinical use.

The trauma of implantation, including the dimpling of local tissue and nerves, may decrease implant recording yield and can cause and/or accelerate glial scarring which isolates the implant from the target tissue. Chronically placed neural penetrating members that remain resident in tissue cause a reactive tissue response, the foreign body response (FBR), involving astrocytes and microglia that result in the formation of a cellular sheath or scar around the penetrating member. The response is highly complex with various chemical signaling pathways, cell types, and damage involved, but overall involves an initial acute phase of glial scarring in response to the initial injury followed by chronic inflammation. The range of applications of neural implants is expanding. However, poor longevity and variable recording quality are frequently points of failure in implant systems. This isolating glial scarring and neural cell loss occurs within 100-500 μm of implant sites.

The FBR limits the clinical potential of chronic neural implants, therefore, minimizing FBR would improve chronic implant performance. Current efforts to minimize FBR include: alteration of array composition and geometry, bio-mimicking coatings, and the creation of floating arrays (i.e., arrays not fixed to the skull) which freely move with the brain; despite these efforts, performance degradation plagues all array types. Bioactive implant coatings or features that can improve host-implant integration and inflammatory mediators such as dexamethasone show short term success, but the long-term effect on neural interface performance after depletion of the bioactive element is unclear.

In one study, implants were engineered to release a brain derived neurotrophic factor (BDNF) analog (Fon D, Zhou K, Ercole F, et al. Nanofibrous scaffolds releasing a small molecule BDNF-mimetic for the re-direction of endogenous neuroblast migration in the brain. Biomaterials. 2014; 35 (9): 2692-2712). The BDNF analog increased neurite growth onto implanted scaffolds and the beneficial effect ended when the BDNF supply was exhausted. A healthy, neural-supportive, anti-inflammatory microenvironment around penetrating electrode arrays may be effectuated by the introduction of increased BDNF, along with other neurotrophic factors. Limiting inflammation has been proven to improve electrode interfaces, as shown in a study of caspase-1 knock-out mice (Kozai T K, Li X, Bodily L M, et al. Effects of caspase-1 knockout on chronic neural recording quality and longevity: Insight into cellular and molecular mechanisms of the reactive tissue response, Biomaterials, 2014; 35 (36): 9620-9634). BDNF has been shown to block the activity of caspase, an enzyme involved in cell death; BDNF at the electrode site may reduce inflammation in a similar way.

Transcranial ultrasound stimulation, such as low-intensity pulsed ultrasound (LIPUS), has been reported to improve behavioral and/or histological outcomes in preclinical models of experimental traumatic brain injury (TBI) and stroke (Su W S, Wu C H, Chen S F, Yang F Y. Transcranial ultrasound stimulation promotes brain-derived neurotrophic factor and reduces apoptosis in a mouse model of traumatic brain injury. Brain Stimul. 2017; 10 (6): 1032-1041); (Chen S F, Su W S, Wu C H, Lan T H, Yang F Y. Transcranial Ultrasound Stimulation Improves Long-Term Functional Outcomes and Protects Against Brain Damage in Traumatic Brain Injury. Mol Neurobiol. 2018; 55 (8): 7079-7089); (Lin W T, Chen R C, Lu W W, Liu S H, Yang F Y. Protective effects of low-intensity pulsed ultrasound on alumimim-induced cerebral damage in Alzheimer's disease rat model. Sci Rep. 2015; 5). The protective effects of transcranial therapeutic ultrasound are likely caused at least partially by enhanced BDNF release from oligodendrocytes and/or astrocytes.

Extending the lifetime of neural implants increases the technology reliability and reduces healthcare costs for patient populations like amputees, which may consist of 3.6 million individuals in the U.S. by 2050. Improved understanding of this technology could also suggest new therapies for TBI and neurodegenerative diseases like dementia. What is missing in the art is a system for applying ultrasound stimulation to the area surrounding an implant. While ultrasound may be known to have positive therapeutic effects, there is no system for directly applying ultrasound to an active neural implant, targeting recording sites of the implant for best results.

While trauma from neural implants and the corresponding FBR is one use case for the application of therapeutic ultrasound, other native non-invasive injuries such as a stroke, epilepsy, percussive force, ischemia, aneurysm, hemorrhage, encephalitis, other TBI, other non-invasive brain injuries, and other tissue injury, whether or not in the brain, may benefit from the application of this therapy. Injury to brain tissue in particular is frequently accompanied by opening of the blood brain barrier and leakage of blood plasma proteins into the brain parenchyma. The presence of plasma proteins within the brain parenchyma activates the resident immunological cells of the brain, microglia, initiating an injury cascade of neuroinflammation, neurodegeneration, and fibrotic encapsulation of the lesion site caused by activated glial cells. This activation of microglia in response to brain and/or central nervous system damage, and the resulting biochemical, physiological and morphological changes induced thereby is known as microgliosis.

In the case of chronically implanted biomedical devices, albumin and immunoglobulins bind to the surface of the implant for encapsulation by activated microglia. This initial protein binding and encapsulating response occurs within minutes to hours of the injury with the acute phase of the injury response peaking approximately 48 hours following injury onset. In vivo imaging of microglia around implanted electrode shanks (measuring 100 μm wide and 15 μm thick) demonstrate injury induced changes in microglia morphology within a nearly 200 μm radius from the implant. (Kozai, T. D. Y., Vazquez, A. L., Weaver, C. L., Kim, S. G., & Cui, X. T. In vivo two-photon microscopy reveals immediate microglial reaction to implantation of microelectrode through extension of processes. Journal of Neural Engineering, 2012; 9 (6)). Further, long term imaging of the chronic glial cell response brain tissue injury has demonstrated tissue morphological changes extending nearly 300 μm from the implant. (Wellman, S. M., & Kozai, T. D. Y. In vivo spatiotemporal dynamics of NG2 glia activity caused by neural electrode implantation. Biomaterials, 2018; 164, 121-133).

Similarly, native or non-invasive injuries may experience tissue morphological changes extending far from the site of injury, as injury response signals are secreted far from the injury site. Microglia are the resident immune cells of the central nervous system and have been shown to alter their morphology and protein expression profiles in response to changes in tissue mechanical properties, presence of blood plasma proteins such as following TBI or hemorrhagic stroke, and tissue ischemia such as during ischemic stroke. In response to these mechanical and biological indicators of tissue injury, microglia migrate towards the injury site and begin secreting inflammatory cytokines such as Interleukin (IL) 1β, IL-6, IL-18, and Tumor Necrosis Factor (TNF) a. These neuroinflammatory signaling molecules are responsible for recruiting circulating macrophages and astrocytes to clear cellular debris and sequester the injury site from healthy tissue through scar formation. While these signals can provide an initial benefit through clearance of cellular debris, prolonged neuroinflammatory cytokine expression has been demonstrated to reduce dendrite complexity of neurons and lead to neuron death near injury sites. Scientific review of molecular mechanisms of microglia neuroinflammation is found for native injury including stroke (Zhang, Y., Lian, L., Fu, R., Liu, J., Shan, X., Jin, Y., & Xu, S. Microglia: The Hub of Intercellular Communication in Ischemic Stroke. Frontiers in Cellular Neuroscience, 2022; 16.) and traumatic brain injury (Nespoli, E., Hakani, M., Hein, T. M., May, S. N., Danzer, K., Wirth, T., Baumann, B., & Dimou, L. Glial cells react to closed head injury in a distinct and spatiotemporally orchestrated manner. Scientific Reports, 2024; 14 (1), 2441.)

What is missing in the art is a system and method for applying ultrasound stimulation to a native injury and the extended tissue area surrounding a native injury. While ultrasound may be known to have positive therapeutic effects, there is no system and method for directly applying ultrasound to an injured tissue area, targeting both the injured area and the extended tissue area for best results.

### SUMMARY

The present invention is directed to devices and methods for delivering acoustic stimulation to the tissue surrounding an implant with one or more electrodes that have been inserted into the tissue. The devices comprise a transducer capable of producing various frequencies of acoustic vibration and an assembly which may retain the transducer and direct the acoustic stimulation in a particular direction, namely, toward an implant. The implant electrode(s) may have one or more recording or stimulating sites thereon along the length of the electrode. The device utilizes a transducer mounted therein to produce acoustic vibrations which are delivered through a chamber having an acoustic coupling medium to target tissue. The device applies a field of acoustic vibrations to areas of tissue directly surrounding the electrode(s), at least at the recording sites thereof. In at least one embodiment, such acoustic vibrations are ultrasonic vibrations; this may also be referred to as acoustic and/or ultrasonic stimulation herein. Ultrasonic stimulation is delivered to the target tissue following insertion of the implant to reduce the body's immune system response to the implant and improve recording at the implant sensors. This response may be characterized as a foreign body response (FBR) and is a result of the insertion and presence of the electrode(s) and implant within the neural tissue.

In at least one embodiment, the implant is inserted on an oblique angle relative to the tissue surface so that the recording site(s) are directly beneath the assembly. In other embodiments, the implant is inserted substantially perpendicular to the surface of the tissue. However, in both embodiments, the implant and the tissue containing the recording sites of the electrode are situated within the field of a transducer capable of producing acoustic stimulation.

The assembly may consist of a series of interconnecting parts placed at the target site of the tissue. In one embodiment, the assembly consists of a base plate having a base aperture, one or more posts, a body, a chamber within the body, and a transducer housing. The assembly is defined along a longitudinal axis which is substantially perpendicular to the tissue plane. A proximal end of the assembly is located along the longitudinal axis closest to the tissue, while a distal end of the assembly is located opposite the tissue. The assembly together with the transducer define the device.

A base plate having a base aperture is positioned on or near target site tissue. The base may be mounted to the skull of a subject, which may be a human, animal, or other being, alive or dead, which may have an implant inserted therein, or directly to the subject's tissue by any mechanism providing a stable and semi-permanent attachment to the subject. The base is positioned around the implant, accommodating the implant, to target the recording sites of one or more implant electrodes. The base includes one or more posts extending parallel to the longitudinal axis of the assembly in the distal direction. The posts are secured to the base so that they may support and retain the remainder of the assembly at the target site. The posts may slidably and releasably retain the body thereon, aligning the two components with each other and with the electrodes and/or recording sites being targeted. The body includes geometrically corresponding post receivers to accept posts of the base when inserted therein. The post receivers accept the posts and align the body and base to place the chamber of the body in communication with the base plate aperture, forming a path for acoustic stimulation.

In some embodiments, a chamber is formed in the body and defined by at least one wall. The chamber retains an acoustic coupling medium, which may be polyvinyl alcohol (PVA) cryogel or other material capable of transmitting acoustic vibrations with minimal dampening or alteration to the frequency of the vibrations. The chamber wall terminates at and defines a chamber aperture toward the proximal end of the assembly and is in communication with the base aperture. The chamber is designed to direct acoustic vibration to the base aperture, and thus to a specific target site of the tissue. Being in communication with both the base aperture and transducer housing aperture, the chamber guides acoustic stimulation to the target site without obstruction.

The body further comprises contours extending parallel to the longitudinal axis toward the distal end to retain the transducer housing and align the housing with the body. An additional contour may consist of one or more alignment members extending from the body to ensure proper alignment between the transducer housing and body. The transducer housing is configured to receive and retain a transducer, such as but not limited to a piezo disc transducer or an annular or ring transducer. Specifically, an aperture formed in the housing receives at least a portion of a transducer therein. The acoustic vibrations discussed herein are produced by the transducer. Small-format, low-cost piezoelectric ceramic disc transducers with resonance near 1 MHz may be used in at least one embodiment. Transducer energy output is ideally kept below the threshold for inducing neural excitation.

In experimental therapeutic use, chronic implants may be placed within a subject from weeks to years. A critical window for treatment occurs within two weeks post-insertion. During this window, therapeutic ultrasound treatments with the above-described device are applied to the target site daily, with decreasing frequency as time progresses. For example, ultrasonic stimulation treatments are administered daily during the first week post-insertion and every other day or every three days in at least the second week post-insertion, preferably for the remainder of the duration of implant residence in the tissue. Treatment in this critical window, also referred to as the acute or early phase, produces better long-term results in experimental subjects. These results allow for better electrode stimulation and better recording of brain activity at the recording sites of the electrode, as shown in FIGS. 18A-18B and as described in more detail below.

To use the device, first an implant is inserted into a subject. This implant may be inserted at an oblique angle as described above. The transducer may have been attached to the housing at any point during the above-described assembly process. Once assembled, the transducer may be selectively activated for limited periods of time to avoid heating the tissue via excess acoustic stimulation. In one exemplary embodiment, the transducer may be activated for periods of 5 minutes, with 5-minute rest periods between activations. This may continue for a period of 15 minutes to complete a treatment cycle, and may be repeated on subsequent days according to the above protocol. The recording sites of the implant are targeted during activation, ideally being at a focal point of the acoustic field. During activation, the recording sites may cease collecting data, as the acoustic stimulation may introduce artifacts into data output.

The ultrasonic field produced by the transducer may be altered by a variety of factors, including but not limited to the geometry of the transducer, frequency of vibration, thickness of the transducer, acoustic lens application focusing the stimulation, concentric annular piezoelectric elements being selectively excited, and by other factors known in the art.

Some embodiments may utilize an annular, or ring-shaped, transducer. The annular transducer, in combination with a correspondingly shaped assembly, allows the body and transducer housing, to define a passage therethrough which allows an implant to be inserted into a subject substantially perpendicular to the tissue. The operation of the device is substantially similar to the operation of the disc-shaped transducer embodiment described further herein. The chamber encircles the passage, forming an annular chamber which may be substantially cylindrical in form, without angling the acoustic field in any particular direction to maximize the overlap between acoustic fields from opposing sides of the annular transducer.

A third embodiment of the present invention comprises a base and a housing. The base having a base aperture is positioned on or near target site tissue. The base may be mounted to the skull of a subject or directly to the subject's tissue by any mechanism providing a stable and semi-permanent attachment to the subject. The base aperture is positioned around the target site being an implant insertion site or a tissue injury area. If present, the base accommodates the implant extending through the base aperture such that the assembly may target the recording sites of one or more implant electrodes. The base includes a base ledge extending from the base aperture substantially perpendicular to the subject tissue surface, a base wall extending from the outer perimeter of the ledge along a longitudinal axis, and a base top surface at the distal end of the base wall. The base wall conforms to the proximal end of the housing such that the housing can be retained within the base wall. The base further comprises a channel in the base wall and a channel opening at the base top surface which is continuous with the channel.

An alignment tab extending from the proximal end of the housing is slidably received and retained within the channel opening and continues from the channel opening to the channel. The channel is sloped such that, when the alignment tab is within the channel, rotation of the housing causes vertical displacement of the housing. A locking mechanism is provided within the base to secure the vertical position of the housing. When assembled, the base aperture is in communication with the proximal end of the housing.

The housing defines a housing lower aperture at its proximal end and contains a horn and transducer therein. The horn is retained within the housing, having a proximal end terminating within the housing aperture and a distal within the housing, the horn being between the transducer and the base aperture. The transducer is mounted to the distal end of the horn and is in contact with the horn. The transducer generates acoustic vibrations when activated. The acoustic vibrations are transmitted through said horn to the subject tissue at the target site, creating an acoustic field in the target site sufficient to reduce tissue injury response in the subject at the target site.

In therapeutic use related to treatment of a native injury, therapeutic ultrasound treatments with the devices and assembly described herein are applied to the target site proximate to the injury, starting immediately following injury with decreasing frequency as time progresses. For example, ultrasonic stimulation treatments are administered daily during the first week post-injury and every other day or every three days in at least the second week post-stimulation, preferably until the body's microgliosis response to the native injury attenuates.

As injury response signals are secreted far from the injury site, subsequent LIPUS treatment should encompass the entire injury site plus an extended volume of tissue beyond the original injury to decrease microglia activation and tissue fibrosis. Indeed, it is critical for the ultrasound application field to encompass the entirety of injured tissue and extended tissue area, as any residual inflammation will continue to evoke a cellular response. Accordingly, the acoustic field should scale with the volume of tissue injury. For example, a single shank electrode measures approximately 1 mm³ while a human stroke measures approximately 10 cm³. Accordingly, the treatment area for such native injuries should be significantly larger. Application of therapeutic ultrasound across extended tissue areas surrounding an injury is critical to treatment of native injuries.

To use the devices and assembly described herein, the base and housing having the transducer are placed proximate to the injury site, likely spaced from the injured tissue and extended tissue area by intervening tissue. The transducer may be selectively activated for limited periods of time to avoid heating the tissue via excess acoustic stimulation. In one exemplary treatment protocol, the transducer may be activated for treatment durations of 5 minutes, with 5-minute rest periods between activations. This may continue for a period of 15 minutes to complete a treatment cycle, and may be repeated on subsequent days according to the above protocol. The native injury site is targeted during activation, ideally being at a focal point of the acoustic field. However, the acoustic field should also encompass the extended tissue area surrounding the injury site where microgliosis response is also occurring.

The device, together with its particular features and advantages, will become more apparent from the following detailed description and with reference to the appended drawings.

### DESCRIPTION OF THE DRAWINGS

FIG. 1 is a perspective view of a first illustrative embodiment of the device of the present invention, having a disc transducer, placed in proximity to neural tissue and showing the placement of an implant electrode relative thereto.

FIG. 2 is an exploded top perspective view of the device of FIG. 1.

FIG. 3 is an exploded bottom perspective view of the device of FIG. 1.

FIG. 4A is a top perspective view of the assembled device of FIG. 1 shown mounted on a stereotaxic frame.

FIG. 4B is a detail view of the assembled device of FIG. 4A.

FIG. 5 is a side cross-sectional view of the device of FIG. 1, showing the chamber and interactions of the posts with the body.

FIG. 6 is a top view of the device of FIG. 1, shown without a transducer, exposing the chamber.

FIG. 7 is an illustrative diagram of an ultrasonic field produced by the device of FIG. 1 with reference to an inserted implant electrode.

FIG. 8 is a diagram of the ultrasonic field shown in FIG. 7 with an adjustment to the field by changing one or more parameters of the transducer.

FIG. 9 is a perspective view of a second illustrative embodiment of the device of the present invention, having an annular transducer, in proximity to neural tissue and showing the placement of an implant electrode relative thereto.

FIG. 10 is an exploded top perspective view of the device of FIG. 9.

FIG. 11 is an exploded bottom perspective view of the device of FIG. 9.

FIG. 12 is a top perspective view of the assembled device of FIG. 9 shown mounted on a stereotaxic frame.

FIG. 13 is a side cross-sectional view of the device of FIG. 9, showing the chamber and interactions of the posts with the body.

FIG. 14 is a top view of the device of FIG. 9, shown without an annular transducer, exposing the chamber.

FIG. 15 is an illustrative diagram of an ultrasonic field produced by an annular transducer of FIG. 9 with reference to an inserted implant electrode.

FIG. 16 is an illustrative diagram of an ultrasonic field produced by an angled annular transducer with reference to an inserted implant electrode.

FIG. 17 is an illustrative diagram of an ultrasonic field produced by concentric annular transducers with reference to an inserted implant electrode.

FIG. 18A are graphical data of implanted electrodes treated with the device and method of the present invention as described in the Example, showing more active recording channels from the treatment compared to controls.

FIG. 18B are graphical data of implanted electrodes treated with the device and method of the present invention as described in the Example, showing increased signal-to-noise ratio from the treatment compared to controls.

FIG. 19 is a perspective view of a third illustrative embodiment of the assembly of the present invention, having a transducer and horn within a housing, shown secured in proximity to neural tissue and an implant electrode by a base.

FIG. 20A is a perspective partial-cross-sectional view of the assembly of FIG. 19, showing the housing separated from the base.

FIG. 20B is a perspective partial-cross-sectional view of the assembly of FIG. 19.

FIG. 21 is a perspective view of the assembly of FIG. 19, showing the housing separated from the base.

FIG. 22 is a side elevational view of the assembly of FIG. 19.

FIG. 23A is a bottom perspective view of the base of the assembly of FIG. 19, having an implant inserted therein.

FIG. 23B is a top perspective view of the base of FIG. 23A.

FIG. 24A is a is a top perspective view of the base of the assembly of FIG. 19.

FIG. 24B is a bottom perspective view of the base of FIG. 24A.

FIG. 25 is a side elevational view of the base of the assembly of FIG. 19.

FIG. 26 is a partially exploded, detail perspective view of the assembly of FIG. 21, having the housing separated from the base.

FIG. 27 is an exploded side elevational view of the assembly of FIG. 19.

FIG. 28 is an exploded top perspective view of the assembly of FIG. 27.

FIG. 29 is a cross-sectional elevation view of the assembly of FIG. 19.

FIG. 30A is a side elevational detail view of a portion of the assembly of FIG. 19, showing the housing in a first illustrative position with respect to the base immediately following insertion of the housing into the base.

FIG. 30B is a side elevation view of the assembly of FIG. 30A, showing the housing in a second illustrative position with respect to the base following a partial rotation of the housing.

FIG. 30C is a side elevational view of the assembly of FIG. 30B, showing the housing in a third illustrative position with respect to the base following further rotation of the housing.

FIG. 31 is a perspective view of the assembly of FIG. 19 connected to a control unit.

FIG. 32 is a top perspective view of the assembly of FIG. 19 shown mounted on a stereotaxic frame.

FIG. 33 is a graphical representation of an example of a treatment protocol according to the methods described herein, showing the ultrasound stimulation cycle, an ultrasound stimulation pulse duration, the repetition rate of ultrasound stimulation pulse durations per second within an activation period, the repetition of activation and rest periods within a treatment session, and the application of a treatment protocol of multiple treatment sessions following injury.

Like reference numerals refer to like parts throughout the several views of the drawings.

### DETAILED DESCRIPTION

As shown in the accompanying drawings, the present invention is directed to a device 100 for delivering acoustic stimulation to an implant 10, having one or more electrodes 12, that has been inserted into tissue 5. The device 100 comprises a transducer 142 capable of producing various frequencies of acoustic vibration and an assembly 102 which may retain the transducer 142 and direct the acoustic stimulation in a particular direction, namely, toward an implant 10, and more specifically to the electrode(s) 12 thereof and at least one recording site 14. The implant 10 electrode(s) 12 may have one or more recording sites 14 thereon along the length of the electrode 12. The device 100 applies a field 160 of acoustic vibrations to areas of tissue 5 in contact with electrode(s) 12, at least at the recording sites 14 thereof, which is referred to herein as the target site 7. In at least one embodiment, such acoustic vibrations are ultrasonic vibrations; this may also be referred to as acoustic and/or ultrasonic stimulation herein. Though described in terms of neural tissue herein for the sake of simplicity, the tissue 5 may be any type of tissue, such as, but not limited to, neural tissue, connective tissue, epithelial tissue, and muscle tissue. In at least one embodiment, the tissue 5 is neural tissue, including but not limited to brain tissue (including cortical and/or deep brain structures), the spinal cord, and peripheral nerves. Tissue 5 may be that of any animal having neural tissue 5, such as but not limited to humans, non-human primates, rodents, rabbits, and other animals used in animal modeling. The device 100 may be mounted directly onto a subject, positioned to capture the recording sites 14 of the implant 10 within its field 160 of ultrasonic stimulation.

Ultrasonic stimulation is delivered to the target tissue 5 following insertion of the implant 10 to reduce the body's response to the implant 10 and improve recording at the implant sensors 14. This response may be characterized as a foreign body response (FBR) and is a result of the insertion and presence of the electrode(s) 12 and implant 10 within the neural tissue 5. FBR is an inflammatory response causing neural tissue 5 damage and glial scarring, reducing the effectiveness of the implant sensors 12. The device 100 of the present invention utilizes a transducer 142 mounted therein to produce ultrasonic stimulation which is delivered to target tissue 7 through a chamber 122 having an acoustic coupling medium 126. Without wishing to be bound by any theory, it is believed that the application of low-power therapeutic ultrasound may induce the release of endogenous brain derived neurotrophic factor (BDNF) from within neural tissue 5. BDNF, an anti-inflammatory neuroprotective factor, along with other neurotrophins, may limit the inflammatory FBR response caused by implant 10 insertion at least in part by blocking caspase, an enzyme involved in cell death.

The device 100 consists of an assembly 102 placed on and/or secured to the body of a subject in proximity to a target site 7 for the acoustic stimulation. This target site 7 is the area of tissue 5 having an implant 10 inserted therein. The implant 10 may consist of one or more electrodes 12 having elongate length and at least one recording site 14 thereon. Specifically, the target site 7 is the electrode 12 and at least one recording site 14 thereof, which may be located anywhere along the length of the electrode 12. In one embodiment, a recording site 14 may be located at a distal tip of the electrode 12. In another embodiment, recording sites 14 may be spaced apart from one another along the length of the electrode 12. These recording sites 14 may measure different aspects of electrical impulses transmitted by the electrodes 12 to the adjacent neural tissue 5 and may collect various data associated with brain activity and such impulses. For instance, in at least one embodiment, the recording site(s) 14 may measure electrical potentials encoding components of neural activity spanning a broad frequency range, including frequencies up to 5 kHz. These electrical potentials may range from low-frequency, large-amplitude, spatially propagating electrical potentials, to local field potentials (LFPs) associated with arousal and behavior, to spatially discrete, high-frequency, single and multi-unit action potentials generated by individual neurons located close to the electrode recording site. Electrical potentials can be recorded simultaneously as a single broadband signal and then components may be individually isolated using common bandpass filtering and feature detection algorithms, creating high dimensional datasets.

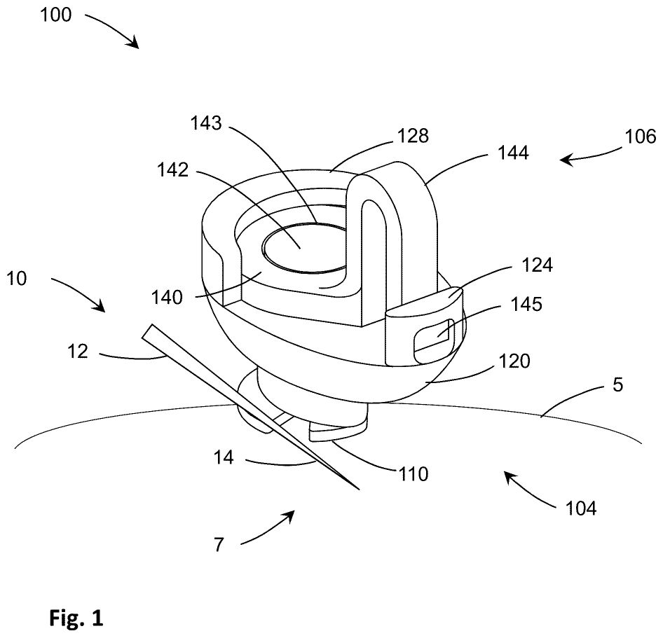

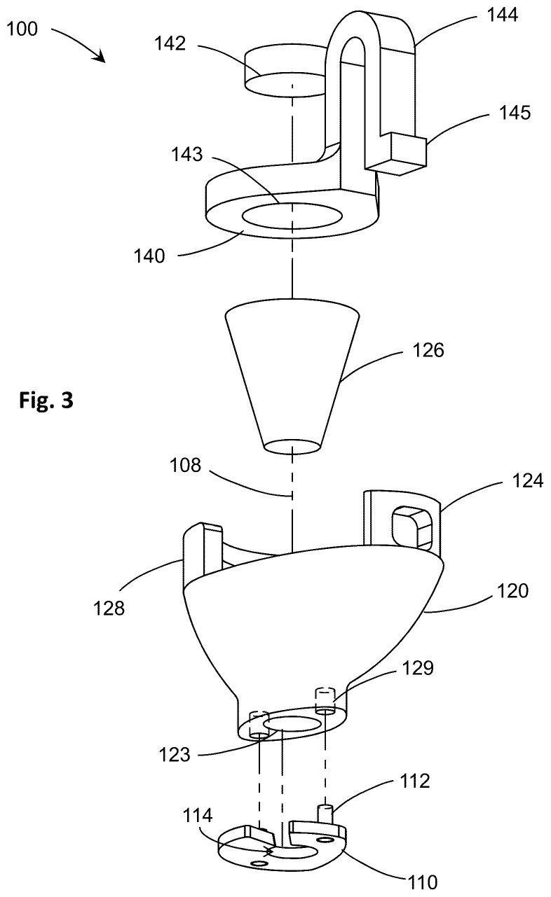

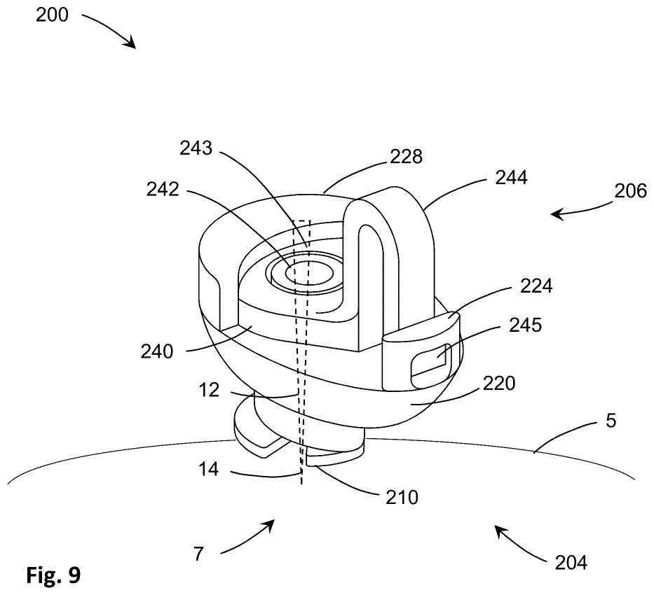

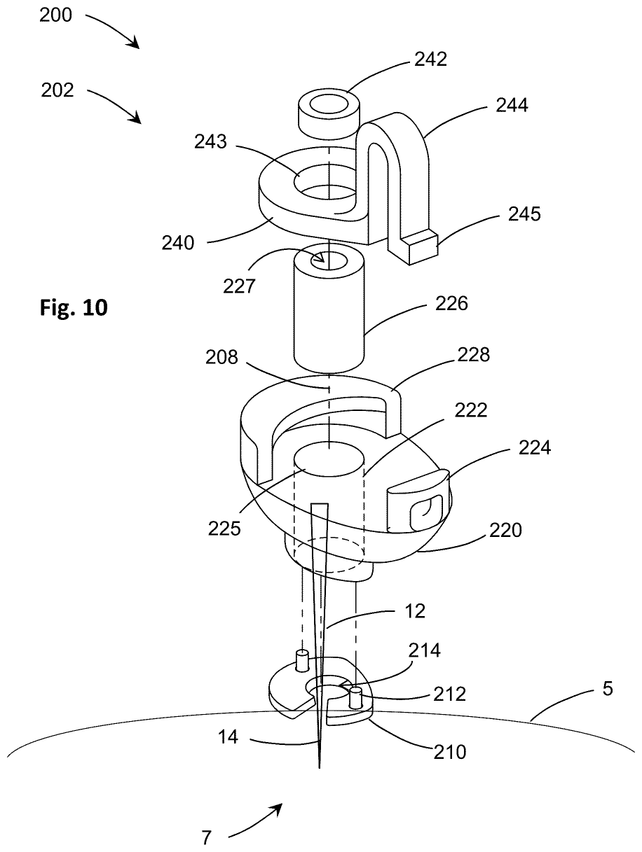

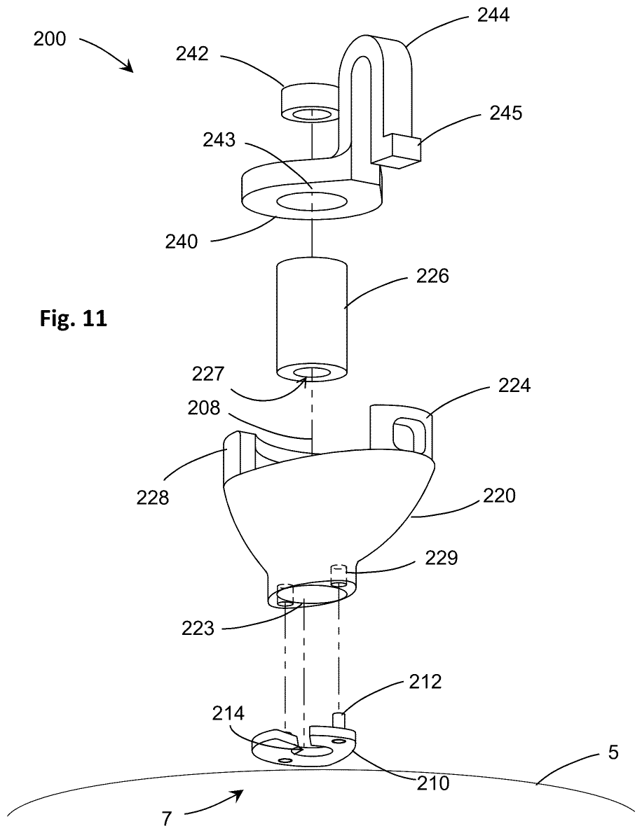

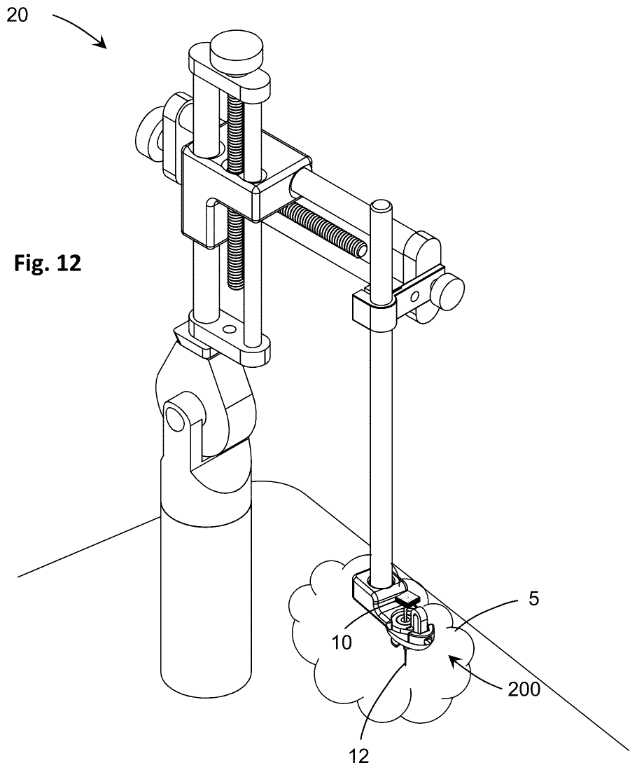

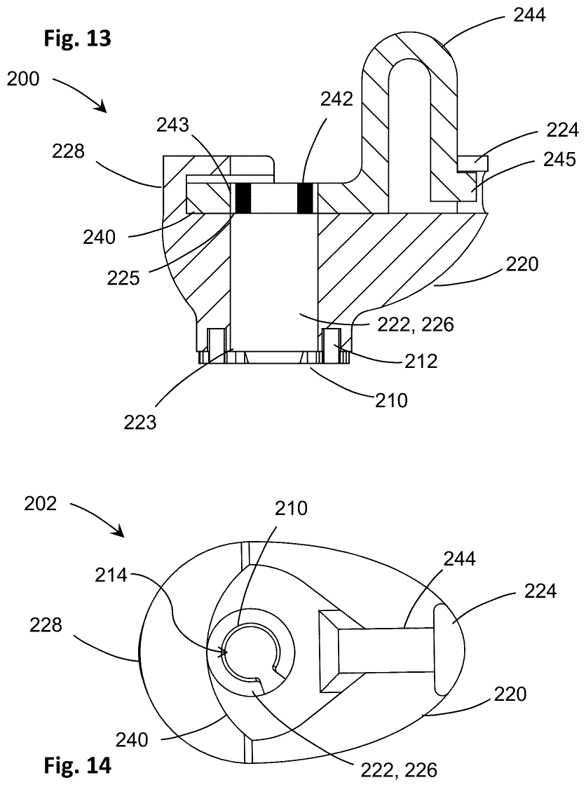

In at least one embodiment, such as shown in FIGS. 1-8, the implant 10 is inserted on an angle so that the recording site 14 sits directly beneath the assembly 102. The implant 10 may be inserted at any oblique angle relative to the surface of the tissue 5, such as but not limited to 5, 10, 20, 30, 40, 45, 50, 60, 70, 80, and 85 degrees. In other embodiments, such as the embodiment shown in FIGS. 9-17, the implant 10 is inserted substantially perpendicular to the surface of the tissue 5. However, in both embodiments, the implant 10 and assembly 102 are situated to place the target site 7, the tissue 5 containing the recording sites 14 of the electrode 12, within the field 160 of a transducer 142 capable of producing acoustic stimulation. Implant 10 electrodes 12 may be placed at any depth relative to the surface of the tissue 5.

Without limitation, a subset of neural implants 10, penetrating intracortical microelectrode arrays 12, are composed of multiple penetrating members with typical cross-sectional diameters in the range of 25-100 μm and are typically implanted 0.25-2 mm into brain tissue 5, but sometimes as deep as several centimeters when targeting deep brain structures in some subjects. The recording sites 14 are relatively small with high impedance (>100 kΩ), a requirement for recording unit activity from individual neurons. Variations in penetrating electrode technologies include insulated metallic microwires, micromachined high density 3-D electrode arrays such as the Utah electrode array (Blackrock Microsystem, Salt Lake City, UT) that are similar in geometry to microwire electrode arrays, and planar thin-film microelectrode arrays like Michigan probes, produced by NeuroNexus Technologies (Ann Arbor, MI), composed of silicon or polymer substrates with multiple electrode sites along the penetrating members. However, the consistency in performance of penetrating neural microelectrode arrays is highly variable. For instance, a group at University of Michigan now has a team of individuals experienced in implanting their microelectrode arrays in subjects, and approximately 67% of the time the implants record unit activity for 3-6 months or more. However, the remaining 33% of the electrode arrays often fail at around 6 weeks, suggesting that if the microelectrode arrays can make it beyond this critical window, they could record neural activity indefinitely. The present device 100 may be used with any of these types of implants 10.

The assembly 102 may consist of a series of interconnecting parts placed at the target site 7 of the tissue 5. In the embodiment shown at FIGS. 1-8, the assembly 102 consists of a base plate 110 having a base aperture 114, one or more posts 112, a body 120, a chamber 122 within the body 120, and a transducer housing 140. The assembly 102 is defined along a longitudinal axis 108 which is substantially perpendicular to the tissue 5 surface. A proximal end 104 of the assembly 102 is located along the longitudinal axis 108 closest to the tissue 5, while a distal end 106 of the assembly 102 is located opposite the tissue, as shown in FIG. 1. The assembly 102 together with the transducer 142 defines the device 100.

### Disc Embodiment

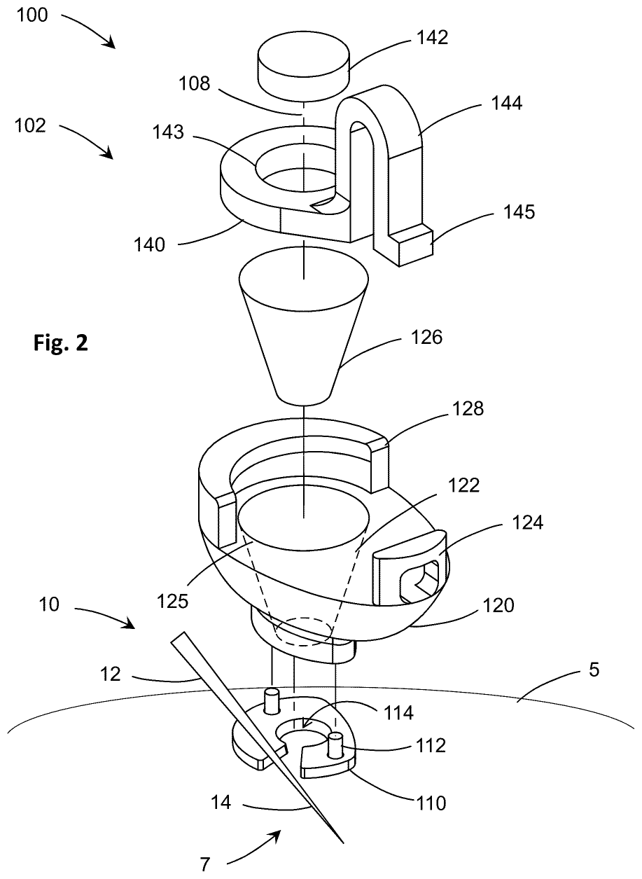

In a first embodiment shown in FIGS. 1-8, and particularly as shown in FIG. 1, the device 100 includes a base plate 110 having a base aperture 114 that is positioned on or near target site 7 tissue 5. The base plate 110 may consist of a plate or any other substantially planar surface and can have any shape suitable for supporting the remainder of the assembly 102. The terms “base” and “base plate” may be used interchangeably herein. The base 110 may be mounted to the skull of the subject or directly to the subject's tissue 5 by any mechanism providing a stable and semi-permanent attachment to the subject, such as but not limited to anchoring by dental acrylic or a similar anchoring substance, by screw attachment, by a combination of dental acrylic and screw attachment, or by any similar mechanism. The base 110 is mounted to the subject at a point where neural tissue 5 is at least partially exposed, having some layers of skin, bone, or other tissue removed to expose the target site 7. In one exemplary embodiment, the base 110 is mounted to the subject via dental acrylic. The base 110 may at least partially encircle the target site 7, at least on the surface above the target site 7. As shown in FIG. 2, the base 110 may have a substantially annular shape defining a base aperture 114 therein. However, in some embodiments, the base 110 does not completely encircle the target site 7, leaving an opening in its substantially annular form to allow access to the site 7 by an implant 10 which may be inserted into the tissue 5 at an oblique angle. The base 110 is positioned around the implant 10, accommodating the implant 10, to target the recording sites 14 of one or more implant electrodes 12.

As shown in FIGS. 1-2, the base 110 includes one or more posts 112 extending outwardly parallel to the longitudinal axis 108 of the assembly 102 toward the distal end 106. In at least one embodiment, the posts 112 are secured to the base 110, though in other embodiments the posts 112 may be integrally formed with the base 110. Together with the base 100, the posts 112 support and retain the remainder of the assembly 102 and properly position the device 100 at the target site 7. Posts 112 may be made of any suitable material for retaining the assembly 102 on the subject but need not be the same material as the remainder of the assembly 102. As shown in FIG. 1, posts 112 may be located on the base 110 on either side of the base aperture 114, positioning the acoustic chamber 122 of the assembly 102 in communication with the target site 7 tissue 5. The posts 112 may slidably and releasably retain the body 120 thereon, aligning the base 110 and body 120 with each other and with the recording sites 14 being targeted. As shown in the embodiment of FIG. 2, the posts 112 may be cylindrical in nature, but in other embodiments may be a projection or contour extending from the base 110 in any geometric shape that is able to align and retain the body 120 thereon.

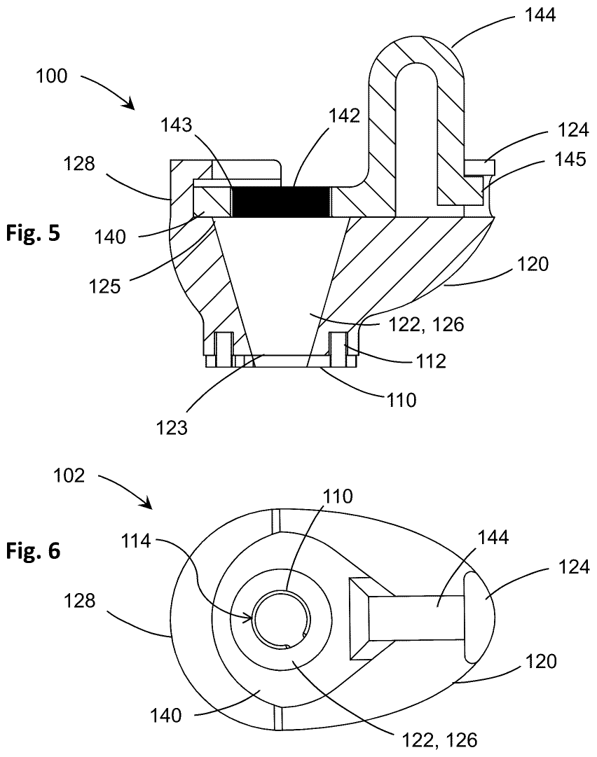

In some embodiments, as shown in FIGS. 1-6, the body 120 is received on and supported by the base 110, aligned properly by the posts 112. The body 120 includes post receivers 129 geometrically corresponding to accept posts 112 of the base 110 when inserted therein. In certain embodiments, the post receivers 129 are matingly configured to the posts 112 and conform to the dimensions thereof. The post receivers 129 accept the posts 112 and align the body 120 and base 110 together to position the chamber 122 of the body 120 in communication with the base plate aperture 114, forming a path for acoustic stimulation transmission, as shown in FIG. 6.

The body 120 includes a chamber 122 formed in the body 120 which is defined by at least one wall 121. The chamber 122 may be cylindrical, conical, or any other shape suitable for holding and retaining material therein and/or directing acoustic stimulation therethrough. The chamber 122 receives and retains an acoustic coupling medium 126 therein, which may be polyvinyl alcohol (PVA) cryogel or other material capable of transmitting acoustic vibrations with minimal dampening or alteration to the frequency of the vibrations. The chamber 122 is capable of retaining acoustic coupling medium 126 in liquid, solid, or semi-solid form such as gels like PVA cryogel. Solid and semi-solid acoustic coupling medium 126 may be formed to conform to the dimensions and shape of the chamber 122, by means suitable for the medium, such as but not limited to by molding, extrusion, 3D printing, milling, and various other techniques. PVA cryogel has mechanical and coupling properties that provide good acoustic coupling for transmission of therapeutic ultrasound. In at least one embodiment, the acoustic coupling medium 126 may be 3D printed conical PVA hydrogel being 10% weight by volume PVA made using two freeze-thaw cycles and having a molecular weight of 78,000 (Polysciences, Inc., Warrington, PA), though other PVA compositions with different weight by volume and molecular weights are also contemplated herein. In some embodiments, the acoustic coupling medium 126 does not fill the chamber 122 but rather lines the chamber. In at least one embodiment, however, an acoustic coupling medium 126 may fill the chamber 122 to transmit acoustic stimulation therethrough. Preferred cone geometry consists of a 3 mm diameter flat cone tip, an 8 mm base, and 10 mm height. However, the cone may have any geometry sufficient to accommodate use of a desired transducer 142. Indeed, in certain embodiments the acoustic coupling medium 126 may be cylindrical in shape, having an outer diameter similar to the inner diameter of the chamber 122.

The chamber wall 121 terminates at and defines a chamber aperture 123 toward the proximal end 104 of the assembly 102 and is in communication with the base aperture 114. In certain embodiments, the chamber aperture 123 and base aperture 114 may have similar or substantially the same diameters. This allows the acoustic coupling medium 126 retained within the chamber to contact tissue 5 through the base 110. The coupling medium 126 may be fitted to the chamber 122, extending between the chamber aperture 123 and a chamber opening 125 defined by the body 120 at its distal end 106. The chamber wall 121 terminates at the chamber opening 125. In some embodiments, the chamber opening 125 and chamber aperture 123 may have similar or substantially the same diameters. In at least one embodiment, as shown in FIGS. 1-8, the chamber aperture 123 may have a smaller diameter than the chamber opening 125. In certain embodiments and as shown in FIGS. 2 and 6, the chamber opening 125 is aligned with the transducer housing 140 when the body 120 and housing 140 are assembled. The chamber 122 may be formed from the coupling medium 126, or the medium 126 may be poured into or otherwise placed within the chamber 122. The chamber 122 is designed to direct acoustic vibration to a specific target site 7 of the tissue 5. Being in communication with both the base aperture 114 and transducer housing aperture 143, the chamber 122 and acoustic coupling medium 126 therein guides acoustic stimulation to the target site 7 without obstruction, as shown in FIG. 6.

The body 120 further comprises contours extending parallel to the longitudinal axis 108 toward the distal end 106 to align and secure the transducer housing 140 to the body 120. For instance, in some embodiments as shown in FIGS. 2 and 5, one contour may be a retention clip receiver 124 extending from the body to receive a portion of the housing 140 therein, such as but not limited to the retention clip 144 having an insert 145 extending therefrom. This retention clip receiver 124 may be formed on the body 120 at any location and may optionally be formed adjacent to the chamber opening 125. The retention clip receiver 124, as shown, defines an aperture through which the retention clip insert 145 may be releasably received, aligning the housing 140 with the body 120. However, the retention clip receiver 124 may consist of any suitable contour or configuration to receive and selectively restrain a portion of the housing 140 retention clip 144 therein. Additional contours may include one or more alignment members 128 extending from the body 120 to ensure proper alignment between the transducer housing 140 and body 120. This alignment member 128 may be formed adjacent to the chamber opening 125 and may be curved, positioned or otherwise configured similarly to at least a portion of the transducer housing 140. As shown in the embodiment of FIG. 2, this alignment member 128 geometrically corresponds to the housing 140 and forms a backstop to align the transducer 140 with the chamber opening 125, and thus with the acoustic coupling medium 126 therein, and to maintain tension between the retention clip 144 and receiver 124 to keep the housing 140 secured to the body 120.

As shown in FIGS. 1-6, the transducer housing 140 is configured to receive and retain a transducer 142, such as but not limited to a disc transducer 142. Specifically, a housing aperture 143 formed in the housing 140 receives at least a portion of a transducer 142 therein. In some embodiments, the transducer 142 may be retained by the housing 140 via frictional fit. In other embodiments, the transducer 142 may be retained by a lip extending from the housing 140, by glue or other adhesive, by screw, clamp or other means sufficient to retain the transducer 142 in the housing 140 during use. The present assembly 102 is dynamic and able to receive transducers 142, 242 with different dimensions and geometry either through a single universal transducer housing 140 or a multitude of transducer housings 140 each adapted to receive a set of transducers 142 having a particular geometry. The housing aperture 143 of the housing 140 aligns with the chamber opening 125 of the chamber 122 within the body 120 such that the portion of the transducer 142 retained in the housing aperture 143 is in communication with and contacting the acoustic coupling medium 126 within the chamber 122. Acoustic vibrations generated by the transducer 140, therefore, may be transmitted to the acoustic coupling medium 126.

In the certain embodiments described above, the housing 140 is selectively attachable to the body 120 by contours on the surface of the body 120 that may correspond to the geometry of the housing, such as but not limited to a retention clip receiver 124 and alignment member 128. As shown, the housing 140 includes a retention clip 144, and retention clip insert 145 extending therefrom, which is configured to align the housing aperture 143, and therefore the transducer 142, with the chamber 123 below. The retention clip 144 is selectively deformable so the housing 140 to be removable from the body 120 when desired. The assembly 102 formed by the base 110, body 120, and housing 140 may be selectively disassembled as needed through the various attachment mechanisms discussed herein, as well as by frictional fit, clips, corresponding contours, or other similar mechanisms. At least a portion of the retention clip 144, such as the arm 146, may be formed of resilient material capable of deforming temporarily to facilitate movement of the clip insert 145 into and out of the retention clip receiver 124. Examples include, but are not limited to, plastics, thermoplastics and polymers of various types.

The device 100 also includes a transducer 142 capable of generating acoustic vibrations when activated. The terms “transducer,” piezoelectric element,” and “piezo” may be used interchangeably herein to refer to a device generating acoustic vibrations when activated. As shown in FIG. 6, the base plate aperture 114 which frames the target site 7 of acoustic stimulation, is in communication with the transducer 142 via the chamber 122, which directs such stimulation to the target site 7. In at least one embodiment, as shown in FIGS. 1, 4, and 5, small-format, low-cost piezoelectric ceramic disc transducers 142 with resonance near 1 MHz may be used (APC International, Ltd, Mackeyville, PA). In other embodiments, as described below, piezoelectric elements having various geometries may be used, such as but not limited to annular and angled piezoelectric elements 242. Transducers 142, 242 used in the device 100 described herein may preferably produce acoustic vibrations of frequencies in the range of 200 kHz and 5 MHz, preferably 500 kHZ-3 MHz, more preferably 1.0-2.2 MHZ, and, in one exemplary embodiment, 1.13 MHz. However, transducers 142, 242 may be used with a range of potential frequencies including up to 2 MHz, up to 5 MHz or values in the tens of megahertz, specifically in the range of 5 and 20 MHz. Regarding transducer 142, 242, the spatial peak temporal average intensity (I_{SPTA}) is preferably equal to 0.5 W/cm². I_{SPTA} being the maximum intensity averaged over the pulse repetition period within the acoustic field 160, indicating the thermal effect of ultrasonic stimulation on tissue 5 (i.e., the amount of heat delivered to target tissue 7 by a transducer 142, 242). The threshold I_{SPTA} value of 0.5 W/cm² has been found to induce BDNF release without crossing neural activation thresholds. Transducer 142, 242 output, ideally may be below a threshold to elicit a brain response to the stimulation, avoiding creating a twitch in the subject. However, other I_{SPTA} value thresholds are contemplated herein, such as but not limited to values in the range of 0.01-2.5 W/cm², preferably 0.1-2 W/cm², and, in one exemplary embodiment, 0.5 W/cm². Transducer 142, 242 voltage may be in the range of 100 and 600 V, preferably above 200 V, or, in one exemplary embodiment, 280 V. Duty cycle percentage in the range of 0.5% and 20%, but preferably near 5%, and, in one exemplary embodiment, 4.2%.

Transducers 142, 242 as described herein may have various geometries which may affect the acoustic field 160 produced by each transducer 142, 242, and therefore vary the stimulation of target tissue 7 with variation of the transducer 142, 242. Transducer 142, 242 diameter may measure in the range of 2 mm-14.5 mm, preferably in the range of 4.9 mm-8 mm, or more preferably 6.4 mm. Pulses generated by the transducer 142, 242 may have durations in the range of 5 ms-200 ms, preferably having 22 ms durations. Transducer 142, 242 thickness may fall in the range of 0.2 mm and 6 mm, preferably 1 mm-2.2 mm. During treatment, the transducer 142, 242 may reach a maximum temperature of 27.6° C., but may ideally run at temperatures below 38.5° C., preferably below 38° C., to avoid tissue damage.

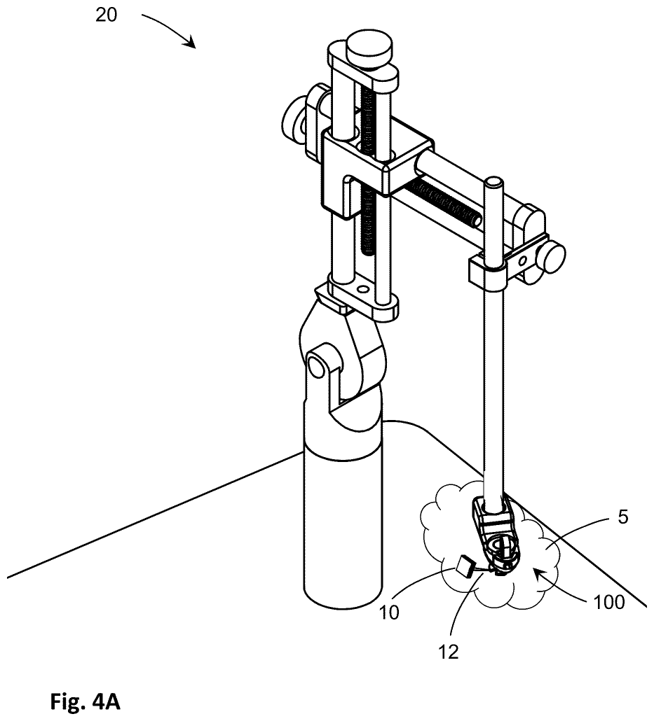

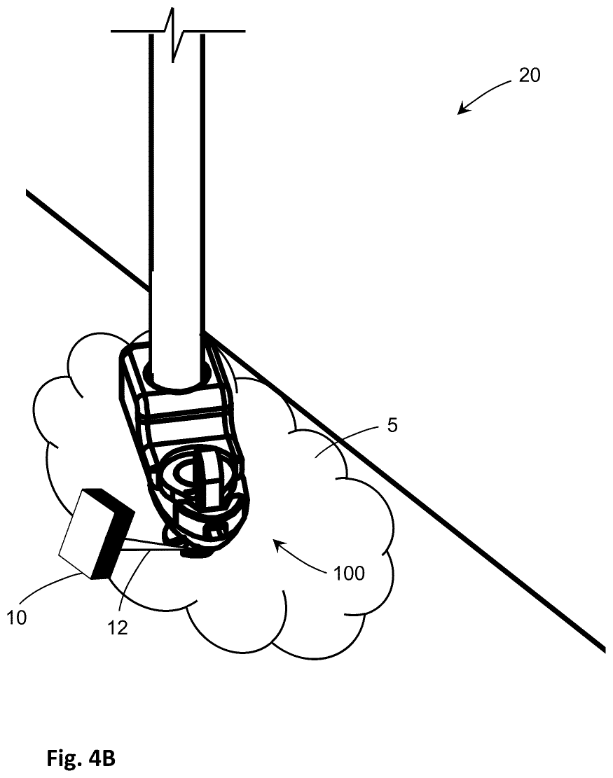

In some embodiments, the device 100, 200 may be mounted to a stereotaxic frame 20 when in use, as shown illustratively in FIGS. 4A-B and 12. A frame 20 may hold the device 100, 200 in proximity to the subject, or the device 100, 200 may be independently mounted to the subject. In some embodiments, the frame 20 may attach to the device 100, 200 via an adapter configured to hold the device 100, 200 by either wrapping around the device 100, 200 or by being inserted between the pieces of the assembly 102, 202 itself, such as by attaching to one or more posts 112, 212 of the assembly 102, 202. In alternate embodiments, the frame 20 may attach by screw, clamp, adhesive, press-fit, or any other similar method. As shown in FIGS. 4A-B, the device 100 may be used in combination with an implant 10 having at least one electrode 12 or an array of electrodes 12. In experimental therapeutic use, implants 10 may be placed within a subject for up to six weeks. A critical window for treatment occurs within two weeks post-insertion, also referred to as the acute or early phase of implant residence. During the first week of this window, therapeutic ultrasound should be applied to the target site 7 daily, with decreasing frequency as time progresses. For example, in a second week ultrasonic stimulation may be administered every other day or every three days.

To use the device 100, first an implant 10 is inserted into a subject. In at least one embodiment, this implant 10 is inserted into tissue 5 at an oblique angle as described above. Importantly, the oblique angle of the implant 10 relative to the assembly 102 allows the transducer 140 to target the recording sites 14 when positioned on the tissue 5, placing the sites 14 within the ultrasonic field 160 generated by the device 100. The location of recording sites 14 along an electrode 12, depth of insertion of an implant 10 and the angle of insertion of the implant 10, allow a user to mathematically determine the target site 7 for ultrasonic stimulation and accordingly attach the base plate 110 to the subject with the base plate aperture 114 aligned with the specific target site 7. In one embodiment, the base plate may be attached directly to the skull of the subject, or may be indirectly mounted to the subject adjacent to the target site 7, as described in further detail above. The base 110 and posts 112 receive the body 120 thereon, slidably retaining the body 120 in alignment with the base aperture 114 so that the chamber 122 and aperture 114 are in communication with one another. The body 120 may or may not be attached to the transducer housing 140 prior to attaching to the base 110. The transducer housing 140 is connected to the body 120, aligning the housing aperture 143 with the chamber 122. The retention clip 144 may be temporarily reversibly deformed by a user to allow the clip insert 145 to slide into the retention clip receiver 124, releasing the clip 144 when the insert 145 and receiver 124 are aligned. The insert 145 and receiver 124 hold the body 120 and housing 140 statically together, aided by the additional contours 128 of the body. The transducer housing 140 may or may not contain the transducer 142 therein prior to attachment to the body 120. In any case, the device 100 may be entirely or partially assembled with the base plate 110 prior to attachment to a subject.

In some embodiments, the transducer 142 may have been attached to the housing 140 at any point during the above-described assembly process. In at least one embodiment, once assembled, the transducer 142 may be selectively activated for limited periods of time to avoid heating the tissue 5 via excess acoustic stimulation. For instance, in one exemplary embodiment, the transducer 142 may be activated for periods of 5 minutes, with 5-minute rest periods between activations. This may continue for a period of 15 minutes to complete a treatment cycle. Other embodiments contemplate different periods of activation and rest, and different overall treatment cycle times, which may be greater or less than those disclosed above. Without limitation, a treatment cycle may have periods of activation for a time in the range about 1 to 15 minutes and periods of rest for a time in the range about 1 to 15 minutes, repeating the periods of activation and rest between 2 to 10 times The recording sites 14 of the implant 10 are targeted during activation, ideally being at a focal point of the acoustic field 160. During activation, the recording sites 14 may cease collecting data, as the acoustic stimulation may introduce artifacts into data output.

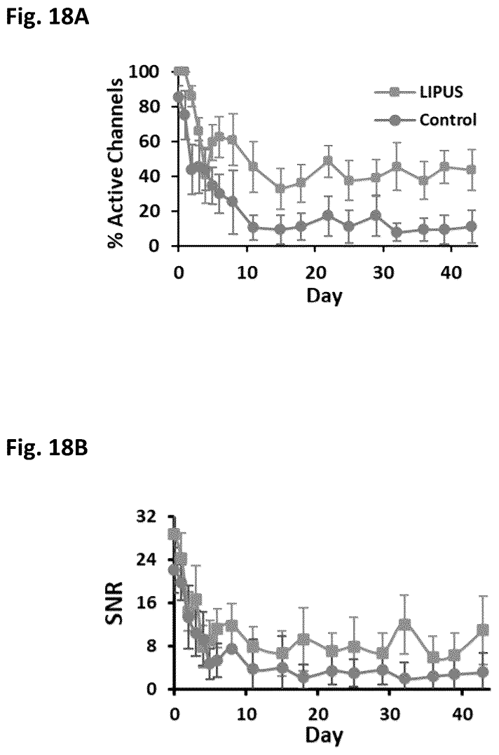

In at least one embodiment, the device 100 may be used to reduce foreign body response in the subject through the following steps. First, the method begins by positioning the device 100 in contact with the tissue 5 and in proximity to the target site 7. Then, the method includes generating acoustic vibrations by activating the transducer 142, 242 for a predetermined period of time, transmitting said acoustic vibrations to the target site 7. Sufficient acoustic vibrations may be applied to the target site 7 to reduce immune system foreign body response in the subject where the electrode 12 contacts the target tissue 7. This may be demonstrated by more active recording channels and/or better signal to noise measurements from recording sites for the duration of the implantation following treatment, such as shown in FIGS. 18A-18B and described in the Example below. The vibrations may be of a frequency and intensity sufficient to stimulate release of at least one endogenous neurotrophic factor in the target tissue 7. In some embodiments, these acoustic vibrations are in the ultrasonic frequency range. Acoustic vibrations may be pulsed, having a duration in the range of about 5 to 200 milliseconds.

In some embodiments, treatment may consist of activating said transducer 142 for a predetermined period of time, turning the transducer 142 on for 5 minutes, then off for 5 minutes, then on for 5 minutes for a total treatment time of 15 minutes. The above steps may be repeated once daily for the first week following implantation of the electrode 12 and once every two or three days during the second week following implantation of the electrode 12. Acoustic vibrations generated during treatment create an acoustic field 160 of said acoustic vibrations at the target site 7, the acoustic field 160 surrounding at least a portion of the electrode implanted in the target tissue 7. This field 160 acoustic field comprises a near field 162 and a far field 164 separated by a transition point 166, where the far field 164 may have a wider diameter than the near field 162. In some embodiments, the field 160 may be modulated by changing the frequency of said acoustic vibrations and the diameter of the transducer 142. However, the field 160 may be modulated by altering any one or more of the above-described operative parameters, such as but not limited to frequency, voltage, temperature, transducer geometry, duty cycle, pulse duration, or I_{SPTA}.

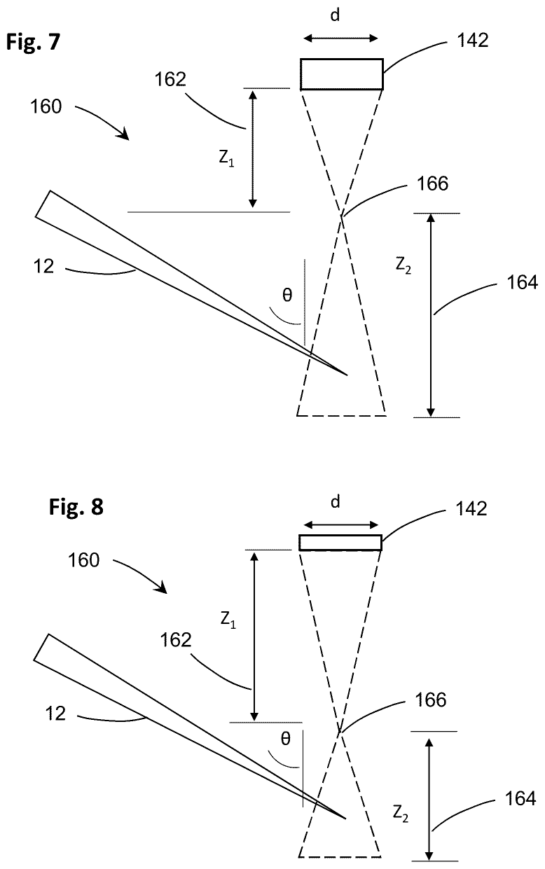

As shown in FIGS. 7-8, the ultrasonic field 160 produced by the transducer may be altered by a variety of factors, including but not limited to the geometry of the transducer, frequency of vibration, thickness of the transducer, acoustic lens application focusing the stimulation, concentric annular piezoelectric elements being selectively excited, and by other factors known in the art. The acoustic field 160 is defined by a near, or proximal, field 162 located adjacent to the transducer 140 and a far, or distal, field 164 located past a transition point 166, penetrating deeper into target tissue 7. The field 160 is approximately the diameter of the transducer 142 within the near field 162 and diverges past the transition point 166 to have increasingly greater diameter than the transducer 140. This divergence from the transition point 166 in the far field 164 is defined by a divergence angle, shown as θ in FIGS. 7-8. Increased diameter of the transducer 140 correspondingly increases the diameter of the near and far fields 162, 166. Resonance frequency of the transducer 142 varies as the transducer 142 thickness varies, where the piezoelectric element 142 operates as a half-wavelength resonator, the frequency of ultrasound produced may be defined by the equation:

$$f = \frac{v}{2 t} \qquad ( 1 )$$

where v is the sound velocity in the piezoelectric element 142 material (often being near 4,000 m/s), and t is the thickness of the piezoelectric element 142. Therefore, a thicker material produces a lower frequency.

### Annular Embodiment

In a second embodiment, the device 200 as shown in FIGS. 9-17, may utilize an annular, or ring-shaped, transducer 242. The annular transducer 242, in combination with a correspondingly shaped assembly 202 shown in FIGS. 9-14, allows the body 220 and transducer housing 240, to define a passage 227 therethrough which allows an implant 10 to be inserted into a subject substantially perpendicular to the tissue 5. The operation of the device 200 is substantially similar to the operation of the disc-shaped transducer 142 embodiment of the device 100 described in detail above.

In certain embodiments, the base plate 210 may be mounted to the subject in substantially the same manner as described above with reference to the first embodiment, accommodating the implant 10 through a base plate aperture 214 therein. Posts 212 extending from the base 210 may be configured to fit within post receivers 229 defined in the body 220 and to receive the body 220 thereon. The body 220 may have a substantially similar configuration to the body 120 described in more detail above, with the exception of a chamber 222 conforming to the contours of the annular transducer 242. In certain embodiments, the chamber 222 containing an annular acoustic coupling medium 226 encircles the passage 227, forming an annular chamber 222 which may be substantially tubular in form, without angling the acoustic field 260 in any particular direction to maximize the overlap between acoustic fields 260 from opposing sides of the annular transducer 242. In some embodiments, the chamber 222 may also be angled, similar to the chamber 122 shown in FIG. 5. The chamber having an annular aperture 223 at the proximal end 204 of the assembly 202, adjacent to the tissue 5, in fluid communication with the base aperture 214. Opposite the aperture 223, a chamber opening 225 at the distal end 206 of the body 220 substantially conforms to the geometry of an annular transducer 242 and is in communication with the transducer 242 when the device 200 is in use. The body 220 has a retention clip receiver 224 and alignment member 228 extending therefrom to receive and restrain the transducer housing 240 and the retention clip 244 insert 245.

A transducer housing 240 in substantially the same form as the disc transducer housing 140, described in more detail above, receives an annular transducer therein 242 and attaches in alignment with the chamber 222 below. A user may reversibly deform the retention clip 244 and place the housing 240 on the base between the clip receiver 224 and alignment member 228.

To use the device 200, first an implant 10 is inserted into a subject. This implant 10 is inserted substantially perpendicularly to the tissue 5 surface. The passage 227 defined by the assembly 202 allows the transducer 240 to target the recording sites 14, placing the sites 14 within the ultrasonic field 260. The location of recording sites 14 along an electrode 12, and depth of insertion of an implant 10 allow a user to mathematically determine the target site 7 for ultrasonic stimulation and accordingly attach the base plate 210 to the subject. The base plate aperture 214 being aligned with the specific target site 7. In one embodiment, the base plate 210 may be attached directly to the skull of the subject, or may be indirectly mounted to the subject adjacent to the target site 7, as described in further detail above. The base 210 having posts 212 receives the body 220 thereon, slidably retaining the body 220 in alignment with the base aperture 214 so that the chamber 222 and aperture 214 are in communication. In other embodiments, the body 220 may or may not be attached to the transducer housing 240 prior to attaching to the base 210. The transducer housing 240 is connected to the body 220, aligning the housing aperture 243 with the chamber 222. A retention clip 244 may be temporarily reversibly deformed by a user to allow the clip insert 245 to slide into the retention clip receiver 224, releasing the clip 244 when the insert 245 and receiver 224 are aligned. The insert 245 and receiver 224 holding the body 220 and housing 240 statically together, aided by the additional contours 228 of the body. The transducer housing 240 may or may not contain the transducer 242 therein prior to attachment to the body 220. In any case, the device 200 may be entirely or partially assembled with the base plate 210 prior to attachment to a subject.

The transducer 242 may have been attached to the housing 240 at any point during the above-described assembly process. Once assembled, the transducer 242 may be selectively activated for limited periods of time to avoid heating the tissue 5 via excess acoustic stimulation. In one exemplary embodiment, the transducer 242 may be activated for periods of 5 minutes, with 5-minute rest periods between activations. This may continue for a period of 15 minutes to complete a treatment cycle. As with the other embodiment, treatment cycles using the annular transducer 242 may be of longer or shorter activation and rest periods or total overall treatment time. The recording sites 14 of the implant 10 are targeted during activation, ideally being at a focal point of the acoustic field 260. During activation, the recording sites 14 may cease collecting data, as the acoustic stimulation may introduce artifacts into data output. In some embodiments, the device 200 may be used and modulated therapeutically by substantially the same methods as the disc-shaped transducer embodiment 100, as described in more detail above. The device 200 may be modulated by changing operative parameters such as but not limited to frequency, voltage, temperature, transducer geometry, duty cycle, pulse duration, or I_{SPTA}.

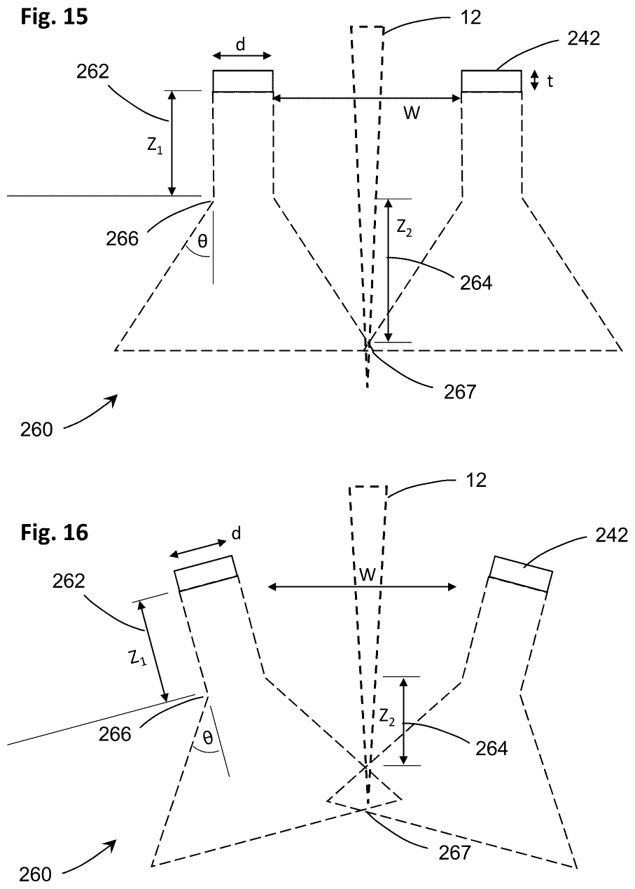

The annular transducer 242 creates a slightly different acoustic field 260 as compared to a disc transducer 142. For instance, taking a longitudinal cross section of the device 200 and annular transducer 242, as shown in FIGS. 15-17, and each side of the transducer 242 may be considered as a single element transducer, acting analogously to the disc-shaped transducer 142 described above. The acoustic field 260 is defined by a near, or proximal, field 262 located adjacent to the transducer 240 and a far, or distal, field 264 located past a transition point 266, penetrating deeper into target tissue 7. The field 260 is approximately the diameter of the transducer 242 within the near field 262 and diverges past the transition point 266 to have increasingly greater diameter than the transducer 240. This divergence from the transition point 266 in the far field 264 is defined by a divergence angle, shown as θ in FIGS. 15-17. The ultrasonic field 260 produced by the transducer may be altered by a variety of factors, including but not limited to the geometry of the transducer, frequency of vibration, thickness of the transducer, acoustic lens application focusing the stimulation, concentric annular piezoelectric elements being selectively excited, and by other factors known in the art.

In some embodiments, the dimensions of the ultrasonic field 260 may be described by a series of equations. Where Z₁ is the length of the near field 262 from the transducer to the transition point 266 and Z₂ is the distance that the far field 264 extends from the transition point 266 to a convergence point 267 of the fields 260 produced by opposing sides of the annular transducer 242:

$$\begin{matrix}{Z}_{1} = \frac{{d}^{2}}{4 λ} & ( 2 ) \\ {Z}_{2} = \frac{W}{2 \cdot tan ( {sin}^{- 1} ( 1.22 λ / d ) )} & ( 3 )\end{matrix}$$

where λ=c/f (“d” being the diameter of each side of the annular transducer, taken at a longitudinal cross-section; “c” being sound velocity in tissue, approximately 1,540 m/s; “f” being the frequency of the ultrasound; “W” being the space between opposite sides of the annular transducer 142, measured from the innermost surface thereof). The distance from the transition point 266 to a convergence point 267 is given by Z₂. This convergence point 267 may define an optimal placement point for a given electrode 12. By way of example and without limiting the disclosure herein, with values f=1.1 MHz, d=2 mm, and W=10 mm, Z₁ would equal 0.71 mm and Z₂ would equal 3.04 mm. Any of these factors may be changed to change the field 260 produced by a given transducer 242. Where d=3 mm and all other parameters remain, Z₁ would equal 1.61 mm and Z₂ would equal 7.22 mm, elongating the field 260 with an increase in “d.”

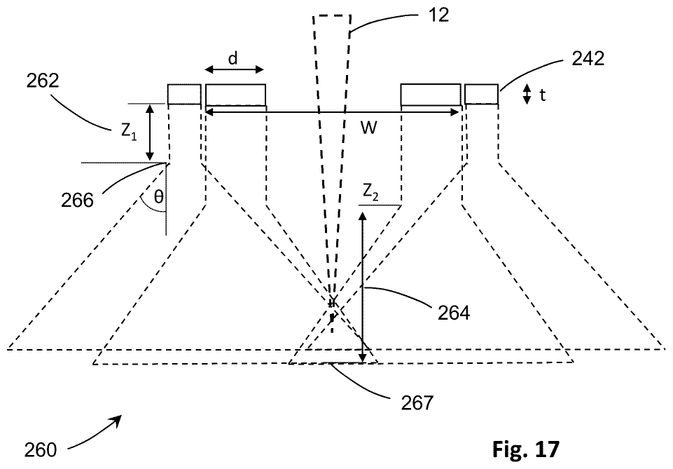

The distance to a convergence point 267, or Z₂, may also be altered by using an annular transducer 242 which has a face at an oblique angle relative to the longitudinal axis 208 of the device 200, as shown in FIG. 16. The fields 260 shown in FIG. 16 may also be produced through the use of a modifier, changing the angle of the field 260. Modifiers may include but are not limited to a lens or wedge. Similarly, as shown in FIG. 17, concentric annular transducer elements 242 may be used in tandem to create overlapping fields 260 with different convergence points 267 which may be optimal for targeting certain electrodes 12.

### Example

To evaluate the effects of low-intensity ultrasound stimulation on long-term neural electrode performance in cortical tissue, an in vivo model was used. Adult subjects (N=10 Sprague Dawley) were implanted with sterile, fixed microelectrode arrays (NeuroNexus, 16 channel 4×4 silicon shanks, 100 μm shank spacing, 125 μm site spacing). Electrode probes were oriented at 45° from horizontal and inserted into cortical layers II/III of the motor or somatosensory cortex using an automated Microdrive to 1.2 mm depth. Subjects were randomly assigned to Stimulation (n=5) or Sham (n=5) treatment groups. During each LIPUS stimulation session, a total of 15 minutes of stimulation was delivered in a periodic fashion to mitigate risk of tissue heating; 5 min ON, 5 min OFF, 5 min ON, 5 min OFF, and 5 min ON. LIPUS and neural recording sessions were conducted daily for days 1-7 post-op and bi-weekly thereafter with subjects lightly anesthetized (0.5-2.0% isoflurane, inhalation) during testing. Electrode impedance measurements and neural signal acquisition (NeuroNexus SmartBox Pro) were taken prior to each LIPUS stimulation session. After six (6) weeks of LIPUS, subjects underwent transcardial perfusion (PBS, followed by 4% paraformaldehyde), and brains were post-fixed, processed and stained for immunohistochemical markers.

The results of these experiments are shown in FIGS. 18A-18B. Specifically, there were insertion-related breakages to the electrode shanks in 2 subjects, leaving n=4 subjects in each treatment group. There were no significant differences in impedance between Control and LIPUS treated groups. Electrophysiology signals were collected via the Allego software package (NeuroNexus) and exported for signal conditioning and spike sorting (SpikeInterface). For each subject, data recorded 5 minutes after the conclusion of LIPUS (or SHAM/Control) treatment was included in this analysis; there were 18 recording days per subject. Interestingly, subjects in the LIPUS Stimulation group demonstrated a significant increase in the percentage of channels that remained active (or had detectible units) after the first week and throughout the rest of the 6-week duration (p<0.0001); ˜ 40% of channels had >1 unit in the LIPUS cohort, while <20% of channels had detectible units in Control subjects. The channels that remained active also maintained a higher signal-to-noise ratio (SNR) over the same time period (p<0.001)

These data show improved electrode longevity using the device and method described herein. Specifically, implanted electrodes treated with the device and method of the present invention showed more active recording channels with better signal-to-noise ratio for the duration of the experiments. In other words, more information was able to be recorded from more neurons for a longer period of time from the electrodes subjected to the treatment described herein than those that were not. This corresponds to a clinically relevant output of a decreased foreign body response (FBR) for the implanted neural devices.

### Additional Embodiment

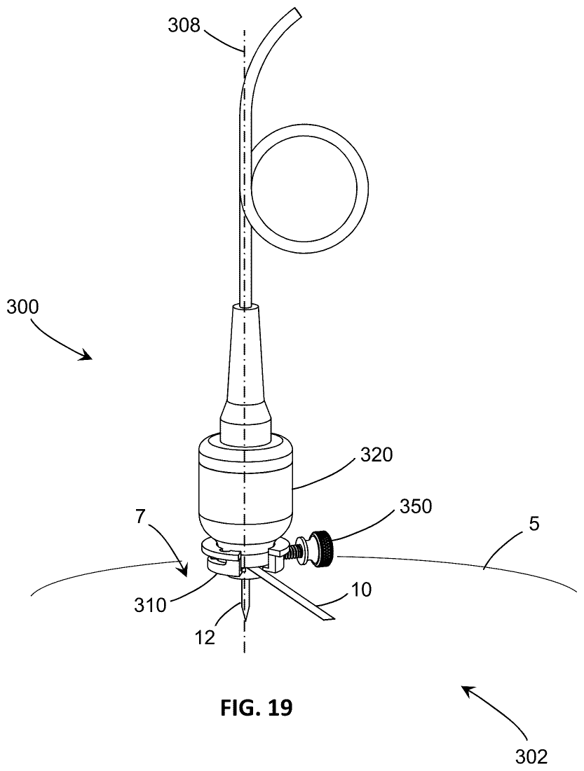

As shown in FIGS. 19-33, a third embodiment of the present invention is directed to a device 300 for delivering acoustic stimulation to a target site 7 of injured tissue 5.

In this third embodiment, most components of the device 300 are integrated into the housing 320. The structure, operation, and methodology of the device 300 is substantially similar to the disc-shaped and annular transducer 142, 242 embodiments of the devices 100, 200 described in detail above. As such, the above description, including, without limitation, the concepts, methods of treatment, methods of operation, ultrasonic fields, operative parameters, and structural features identified above, may apply equally to this third embodiment of the device 300.

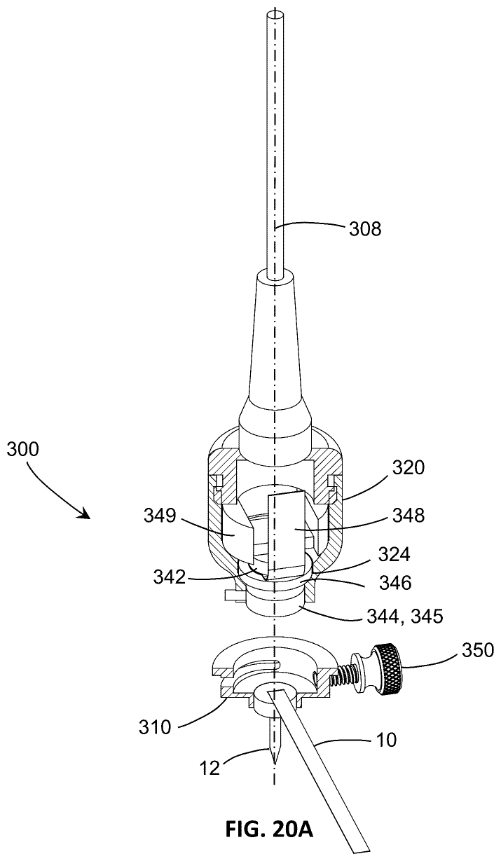

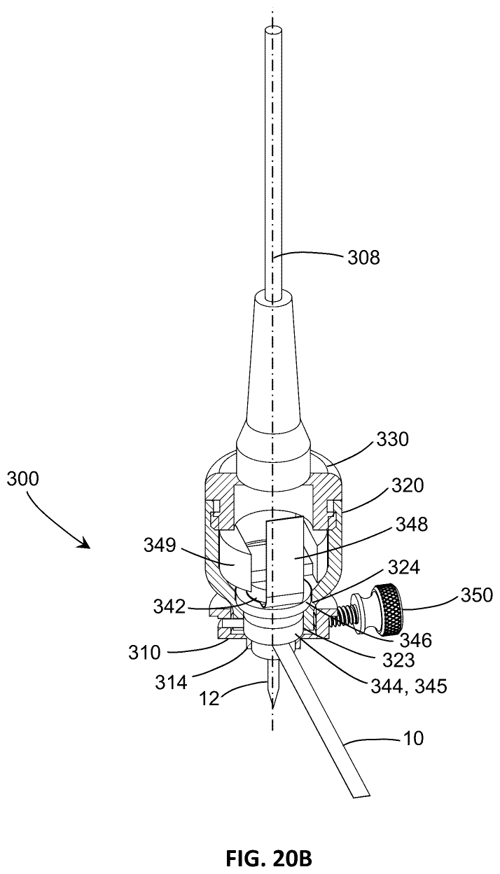

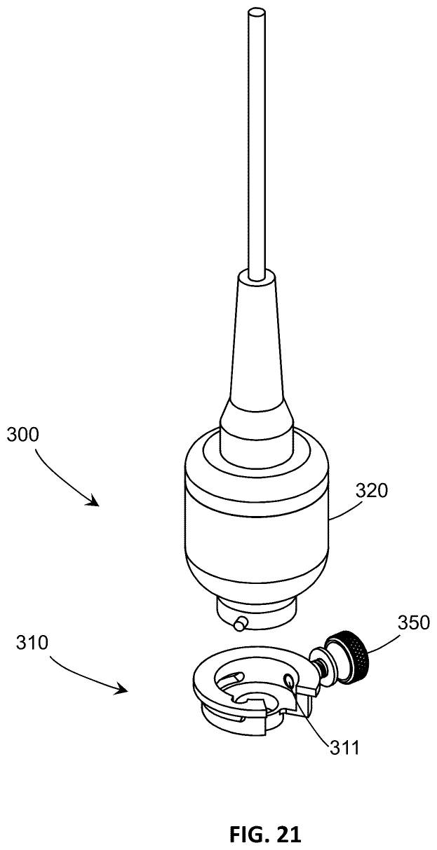

As shown in FIG. 19, the assembly 302 is defined along a longitudinal axis 308 which is described herein as substantially perpendicular to the injured tissue 5 surface, though other angles of approach, including oblique and acute angles, are also contemplated. A proximal end of each constituent piece of the assembly 302 is located along the longitudinal axis 308 closest to the tissue 5, while a distal end of each constituent piece of the assembly 302 is located opposite the tissue. The assembly 302 is primarily comprised of the device 300 and a base 310. The device 300 generates and applies acoustic vibrations to areas of tissue 5 at the target site 7 which may be in contact with electrode(s) 12 of an implant 10, when present, and as described further above, and may be used with any of the types of implants 10 described herein. The device 300 features a housing 320 which retains a transducer 342 to generate acoustic stimulation and an acoustic transmission horn 344 to direct the acoustic stimulation in a particular direction, as shown in FIGS. 20A-21. The device 300 may be mounted onto a subject via the base 310, which is positioned proximate to a target site 7.

As used with reference to this third embodiment, “injured tissue” means collectively both the tissue receiving an injury (also referred to as the “damage locus” or “site of injury”), and the volume of surrounding tissue affected by the injury (also referred to as the “affected tissue”). The damage locus may be from a foreign body such as an insertion of an implant 10 or electrode 12 in the case of an invasive injury, or from non-invasive injury such as but not limited to stroke, epilepsy, percussive force, ischemia, aneurysm, hemorrhage, encephalitis, and other trauma-induced tissue injuries. The affected tissue is the tissue experiencing the biochemical, physiological and morphological cascade induced by such injury, including but not limited to microgliosis, FBR and their downstream effects. Though injured tissue 5 is described herein in relation to brain or neurological tissue, the devices and methods described herein may be used to treat injury and cellular responses to injury in other tissues as well.

As used with reference to this third embodiment, the “target site” means the tissue targeted by the acoustic field energy generated by the device 300 as described herein. The target site 7 includes at least a portion of the damage locus, and preferably includes the entire injured tissue area, regardless of whether the injury is invasive or non-invasive.

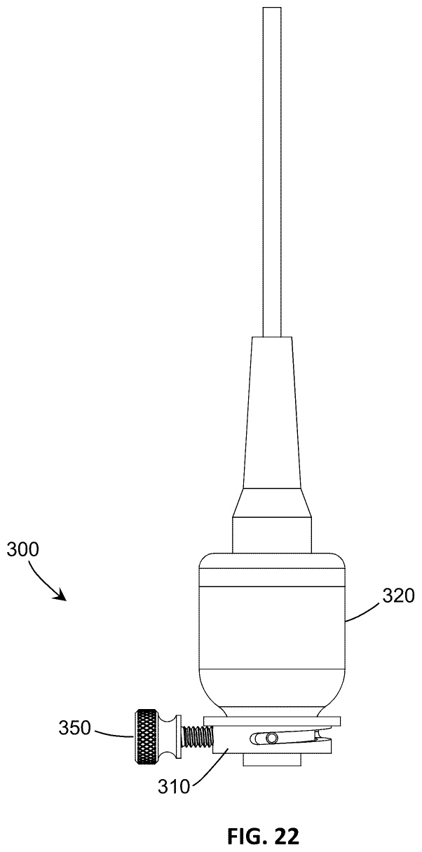

The present assembly 302 is designed to provide for equal loading of the horn 344, meaning that the horn 344 is level with respect to the base 310 and the polymer 345 described herein is equally compressed, near the target site 7 for lossless and equal transmission of ultrasonic energy generated by the transducer 342. It is known within the field that changes in axial and radial loading forces on the transducer 342 will alter impedance and ultrasound resonance frequency. To standardize the load on the device 300 during application of therapeutic treatment, the present base 310 and housing 320 of this third embodiment incorporate an interlocking mounting mechanism therebetween, with variable adjustment of the distance between the base 310 and housing 320 along a predefined continuum, interlocking as shown in FIGS. 20B and 22 and described herein. The base 310 serves as an alignment guide and mounting point for the application of the housing 320. The terms “base” and “base plate” may be used interchangeably herein.

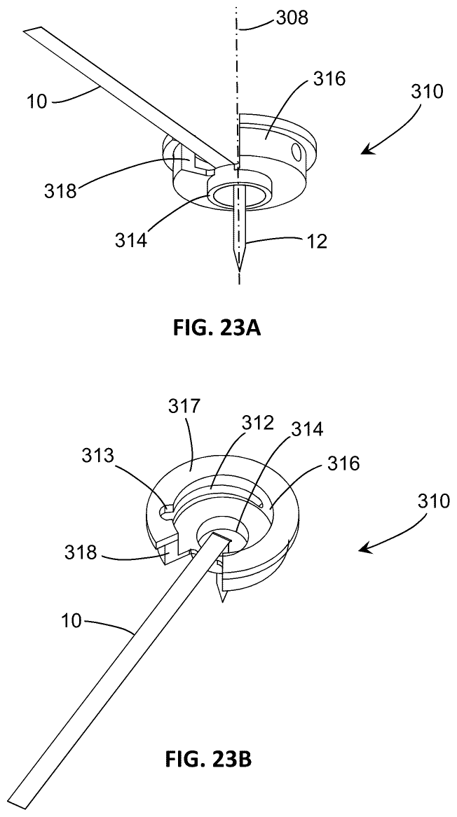

As shown in FIGS. 23A-26, the base 310 defines a substantially annular base aperture 314 that, when mounted onto a subject, is positioned on or near target site 7 tissue 5. For embodiments including electrodes 12 and/or implants 10, the base 310 is placed on or near the location of electrode 12 insertion site and/or implant 10 residence. For transcranial applications such as when treating native injuries, the base 310 may be applied to the surface of a subject's skin as close to the site of injury as is feasible. As used herein, “native injuries” refers to non-invasive injuries such as a stroke, epilepsy, percussive force, infarction, aneurysm, ischemia, hemorrhage, encephalitis, spatially confined neurotoxic cell death, neuron hyper-excitability, vascular reperfusion, other TBI, other non-invasive brain injuries, or other tissue injury, whether or not in the brain, and the microgliosis or other immune cell response associated with such injuries. In such applications, the base 310 is placed on the surface near the site of injury, ideally capturing extended tissue area being affected by microgliosis to address the entire injured tissue 5. As discussed herein, methods and devices for targeting injury at target sites 7 proximate to an implant 10 may be equally applied to target sites proximate to other tissue injury. Skin, bone, skull, muscle, blood vessels, adipose tissue, and the like, may be present between the base 310 and target site 7, and are referred to as “intervening tissue” herein. Acoustic stimulation must pass through intervening tissue before it can reach the target site 7 tissue 5.

The base 310 is constructed from biologically compatible metals such as aluminum, titanium, or stainless steel or any suitable material for retaining the device 300 on the subject. The base 310 may be mounted to the skull of the subject or directly to the subject's tissue 5 by any mechanism providing a stable and semi-permanent attachment to the subject, such as but not limited to anchoring by dental acrylic, epoxy, or a similar anchoring substance, or by any other suitable mounting mechanism. Optionally, a screw attachment may aid in mounting the base 310 to the subject. The base 310 is mounted to the subject via the base aperture 314 either at a point where neural tissue 5 is at least partially exposed, having some layers of skin, bone, or other tissue removed to expose the target site 7, or, in embodiments for treatment of a native injury, at a point on the outer surface of a subject (such as the head) where intervening tissue is present between the base 310 mounting site and the target site. In one exemplary embodiment, the base 310 is mounted to the subject via dental acrylic. The base aperture 314 is sufficiently dimensioned to correspond to the width of at least part of the target site 7, such as at least the damage locus, more preferably covers the damage locus and at least a portion of the surrounding affected tissue, and most preferably covers the entire injured tissue. Accordingly, in some embodiments, the base aperture 314 may have a diameter that corresponds to or is larger or smaller than the diameter of the damage locus, but in at least one embodiment is substantially the same diameter as the widest part of the damage locus. In certain embodiments, the base aperture 314 may have a diameter that corresponds to or is larger or smaller than the diameter of the target site 7, but in at least one embodiment is substantially the same diameter as the widest part of the target site 7. In a preferred embodiment, the base aperture 314 fully encompasses the target site 7 by encircling the surface above the target site 7 and has a height sufficient to form a well, which in certain embodiments is an adhesive well, therein.

The well is a reservoir which retains a mounting material used as an adhesive to anchor the base 310 to the subject and to anchor the implant 10 to the subject, when present. The height of this well is provided by an elevational stand-off of the remainder of the base 310 from the skull or tissue 5 of the subject. However, in alternate embodiments without an implant 10 present, an elevational stand-off may not be necessary. This height is designed to match the transmission wavelengths of the chosen mounting material in order to maximize ultrasonic energy transmission to the implant 10, such that the ultrasound transmissions are not out of phase, and preferably are in phase, when hitting the target site 7.

In embodiments having an implant 10, the base 310 is preferably mounted to the subject prior to implant 10 insertion. The base 310 allows the implant 10 to access the target site 7 by passing through the base aperture 314, as shown in FIGS. 23A and 23B. This insertion process may accommodate placement of the implant 10 within 30 degrees of variation from the longitudinal axis 308. An electrode cabling pass-through aperture 318 is defined by the base 310 and accommodates the implant 10 cabling passing from the base aperture 314 to the environment beyond the base 310. In a preferred embodiment, the pass-through aperture 318 allows the implant 10 cabling to pass above the base aperture 314, however, in alternate embodiments this pass-through aperture 318 may be provided as a break within the base aperture 314 itself; this structure may be advantageous in accommodating an existing implant 10. Alternatively, the base 310 may not include a pass-through aperture 318 in embodiments where an implant 10 is not used. The base aperture 314, and accordingly the base 310, may vary in size to encompass an electrode insertion site or a native injury damage locus ranging in size from 1 mm²-10 cm², encompassing applications such as rodent model organisms on the lower end and human applications on the higher end.

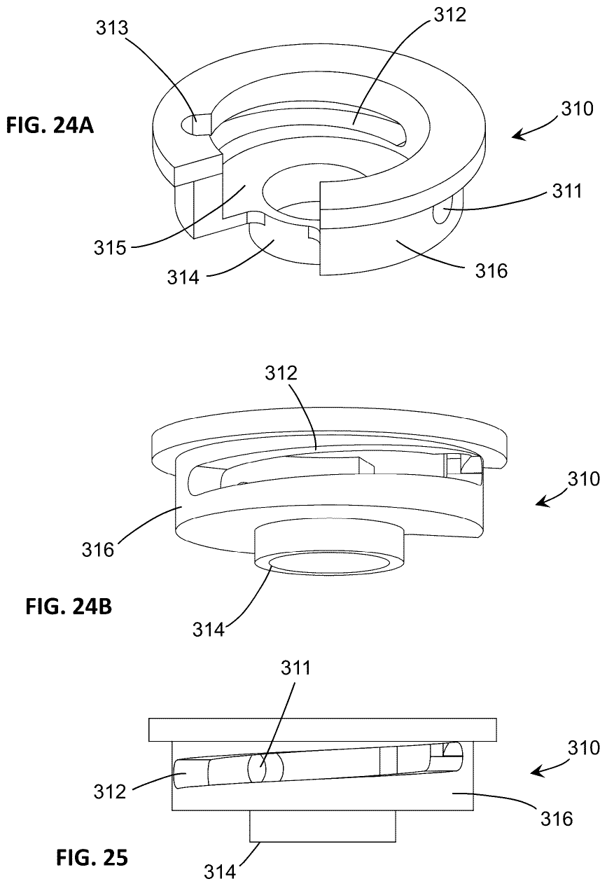

The base 310 further comprises a ledge 315 extending from the distal portion of the base aperture 314 transverse to the longitudinal axis 308, shown in FIGS. 24A-25. A wall 316 extends from the outer perimeter of this ledge 315 along the longitudinal axis 308 toward the distal end of the base 310 and is sized to conform to the proximal end of the housing 320 when the housing 320 is secured within the base 310. The outer diameter of the housing 320 is therefore less than the inner diameter of the wall 316 such that the housing 320 can fit within the wall 316 and attach to the base 310. For instance, each of the outer diameter of the housing 320 and inner diameter of the wall 316 being approximately ½ inch in diameter in at least one embodiment. The close fit provided by this configuration axially stabilizes the housing 320, which rests on or near the ledge 315 and is bounded by the wall 316 when secured within the base 310.

Similar devices face the problem of proper surface mating between the housing 320 components, particularly the horn 344, and the base 310. Variation in thicknesses of mounting material within the well formed by the base aperture 314 and other similar factors may require axial adjustment of the housing 320 with respect to the base 310, such as along the longitudinal axis 308. Particularly, millimeter-level adjustment of the housing 320 may be needed to ensure proper contact between the mounting material and the horn 344 and to achieve the desired resonance frequency depending on the material and amount thereof used. Accordingly, the base 310 includes an adjustment mechanism which allows for fine adjustment of the housing 320 along the longitudinal axis 308 of the assembly 302. This adjustment mechanism may or may not be necessary in embodiments when no implant 10 is utilized. To provide this mechanism, the base 310 further comprises a channel 312, a channel opening 313, and a receiver 311, each being formed within the wall 316. As part of this mechanism, and as described in greater detail below, the housing 320 includes an alignment tab 329 extending therefrom. As shown in FIG. 25, the channel 312 is an elongate opening through the wall 316, spanning a portion of the circumference of the wall 316 from the channel opening 313, and is sloped.

As shown in FIGS. 23B, 24A and 26, the channel opening 313 is provided at a top surface 317 of the base 310 which may be the top surface of the wall 316, being at least the depth of the wall 316, or which, in a preferred embodiment, extends radially outward transverse to the longitudinal axis 308 farther than the wall 316 such that it may fully accommodate the alignment tab 329 through the channel opening 313 and support the portion of the wall 316 surrounding the channel 312. Each of the channel 312 and opening 313 are correspondingly sized to receive and accommodate the alignment tab 329 slidingly therethrough. In a preferred embodiment, the channel 312 spans a quarter of the circumference of the wall 316, allowing for a quarter turn of the alignment tab 329, and therefore the housing 320, when assembled, but may span any portion of this circumference to allow for greater or lesser rotation of the housing 320. In alternate embodiments, this configuration may be mirrored and maintain substantially similar function, wherein the alignment tab 329 may be located on the base 310 while the channel 312 and opening 313 are located on the housing 320.

The channel 312 begins at the channel opening 313 and extends as described above. In a preferred embodiment, as shown in FIG. 25, the channel 312 slopes downward from the channel opening 313 at an angle in the range of 1 to 10 degrees, preferably a 3-degree angle, relative to the base aperture 314. The end of the channel 312 farthest from the channel opening 313 serves as a backstop for the housing 320 rotation. In the preferred embodiment, this allows only a quarter turn of the housing 320 relative to the base 310. Because the channel 312 is elongate, it allows for continuous movement of the alignment tab 329, and therefore, axial adjustment of the housing 320 along the continuum defined by the channel 312. In alternate embodiments, the channel 312 may slope upward from the channel opening 313 or may descend or ascend in a stepwise fashion rather than as a continuous slope shown in the foregoing Figures. The slope of the channel 312 may also be eliminated if adjustment of the housing 320 relative to the base 310 is not desired.

In a second embodiment of the base 310 not shown in the Figures, the method of attachment to the housing 320 differs. In this second embodiment, the alignment tab features and locking mechanism are replaced by a guide column extending from the top surface of the wall toward the distal end of the assembly. Two projections extend from the housing in lieu of the alignment tab, sized to create a recess therebetween which receives the guide column. The projections rest on the top surface of the wall when the housing is fully inserted into the base, maintaining alignment of the base and housing during operation of the assembly.

The receiver 311 accommodates a locking mechanism 350 therein, which secures the housing 320 within the base 310. In the exemplary embodiment shown in FIGS. 19-22 and 26, the locking mechanism 350 is a thumbscrew which is received within a threaded receiver 311 opposite the channel 312. The thumbscrew 350 is rotated to threadably engage with the threaded receiver 311 and secure against a portion of the housing 320 by passing through the receiver 311. This locking is held in place by frictional engagement of the thumbscrew 350 and receiver 311 threading. This locking prevents further rotation of the housing 320 relative to the base 310 and therefore prevents further adjustment of the housing 320 via the channel 312. In alternate embodiments, the locking mechanism 350 may be other types of pins similar to a thumbscrew, a spring plunger, set screw, or other suitable mechanical locking mechanism. Alternatively, a frictional fit between either the base 310 and housing 320 or the alignment tab 329 and channel 312 may be sufficient to secure the housing 320 relative to the base 310. Further, the alignment tab 329 itself could serve as the locking mechanism 350 by rotating or otherwise moving to engage with the surface of the channel 312, securing the housing 320 mechanically or by frictional fit. Vibrational movement of the housing 320 within the base 310 during operation would be minimal, on the order of tens of nanometers; accordingly, these described locking mechanisms sufficiently secure the housing 320.

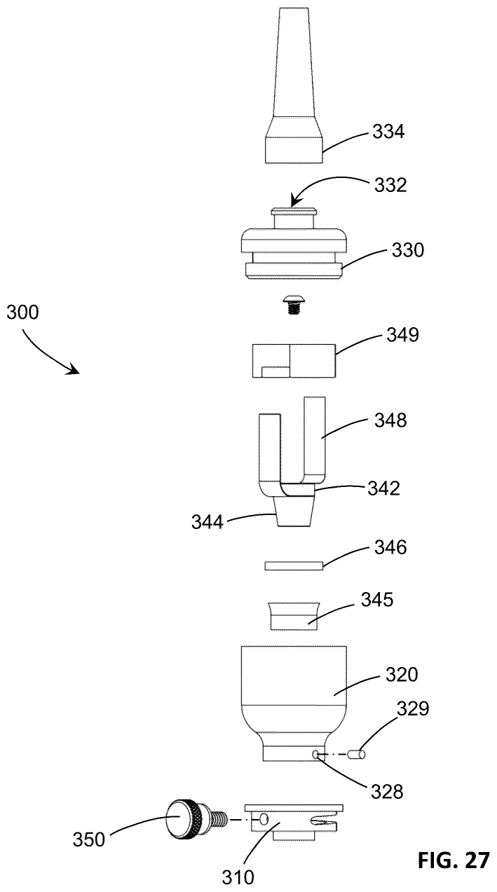

As shown in FIGS. 27-29, the housing 320 retains the horn 344, a horn alignment member 346, the transducer 342, a transducer flexible circuit 348, and ground 349 in its interior, with the alignment tab 329 extending from the exterior of the housing 320 at its proximal end. The housing 320 is fabricated from a thermally stable, non-porous material allowing for use of medical and laboratory sterilization methods, these materials include metals such as titanium, aluminum, or stainless steel, other robust materials such as engineering polymers, or any other material which is capable of encapsulating the various components of the device 300, allowing for surface cleaning, and preventing soil and fluid ingress.

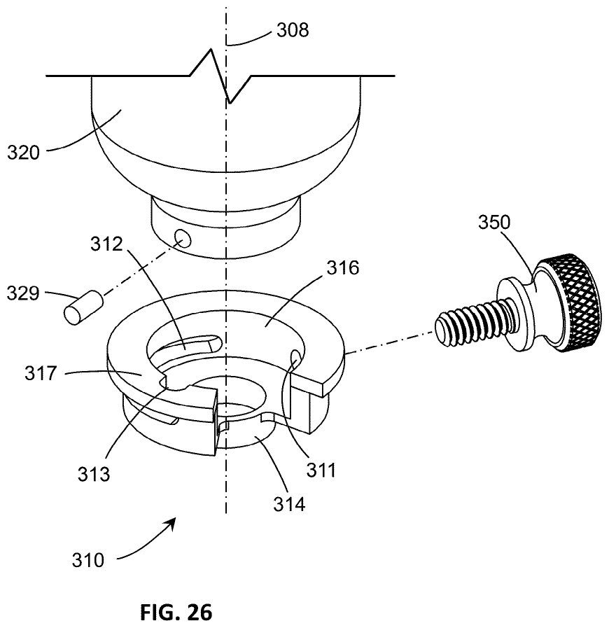

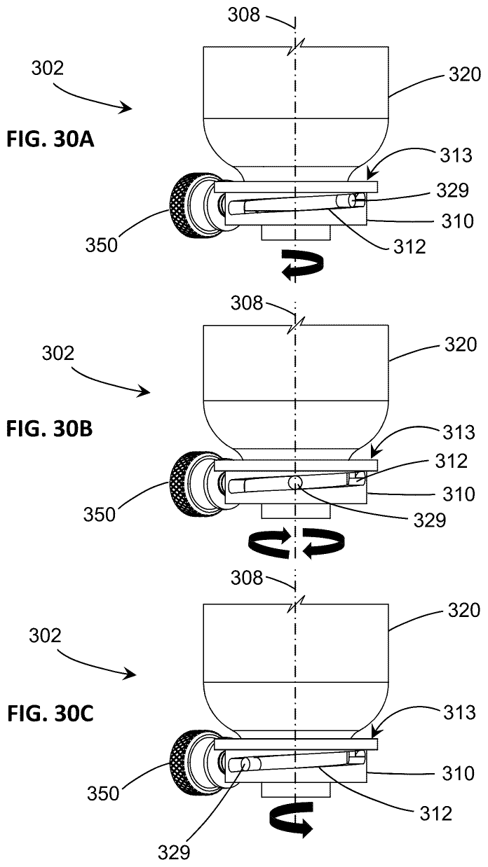

In a preferred embodiment, the alignment tab 329 is a dowel which is securely affixed to the housing 320 by insertion into an alignment tab recess 328 at the proximal end of the housing 320 by frictional fit, adhesive, or other means of secure attachment. The dowel 329 measures approximately 1/16 inch in diameter by ⅛ inch in length. In alternate embodiments, the alignment tab 329 is one of unitary construction with and extends from the exterior of the housing 320. As shown in FIG. 26, when inserting the proximal end of the housing 320 into the distal end of the base 310, the alignment tab 329 must be in registry with the channel opening 313 such that when the housing 320 is placed onto the base 310, the alignment tab 329 enters the channel opening 313 and proceeds along the longitudinal axis 308 into the channel 312. The length of the alignment tab 329 extends transverse to the longitudinal axis 308, such that it at least partially occupies the depth of the channel 312, preventing the housing 320 from moving along the longitudinal axis 308 without a corresponding rotational movement about such axis, moving the alignment tab 329 down the slope of the channel 312, as shown in FIGS. 30A-30C. Therefore, the alignment tab 329 is at least as long as a portion of the depth of the wall 316 through which the channel 312 extends, and in a preferred embodiment is at least as long as the thickness of the wall 316.

The alignment tab 329 is adjusted along the channel 312, by rotation of the housing 320, until the desired displacement of the housing 320 is reached. Particularly, FIG. 30A shows the assembly 302 wherein the alignment tab 329 is at a first position in the channel 312 immediately following insertion of the housing 320 into the base 310. At this point, the housing 320 may be moved along the longitudinal axis 308 away from the base 310, as the alignment tab 329 may still move through the channel opening 313, or the housing 320 can be rotated, moving the alignment tab 329 along the channel 312. FIG. 30B shows the assembly 302 wherein the alignment tab 329 is at an example second or intermediate position in the channel 312 following a partial rotation of the housing 320 about the longitudinal axis 308. Due to the slope of the channel 312, this rotational movement of the housing 320 causes a corresponding movement of the housing 320 toward the proximal end of the assembly 302 along the longitudinal axis 308. Though only one placement is shown for the second or intermediate position, it should be appreciated that there may be any number of intermediate positions along the continuum of the channel 312 between the first initial position and the third final position. FIG. 30C shows the assembly 302 wherein the alignment tab 329 is at a third position in the channel 312 following further rotation of the housing 320 about the longitudinal axis 308, causing further movement of the housing 320 toward the proximal end of the assembly 302 along the longitudinal axis 308. At this third position, the alignment member 329 is at the end of the channel 312 opposite the channel opening 313; rotational movement of the housing 320 about the longitudinal axis 308 is only possible if moving back up the channel 312 toward an intermediate position. The housing 320 may be removed from the base 310 by reversing the foregoing movements. Following adjustment of the alignment tab 329 to the desired position, the locking mechanism 350 is engaged, as described above, to maintain the position of the alignment tab 329. In alternate embodiments, as described above, the locking mechanism 350 may be integrated into the alignment tab 329.

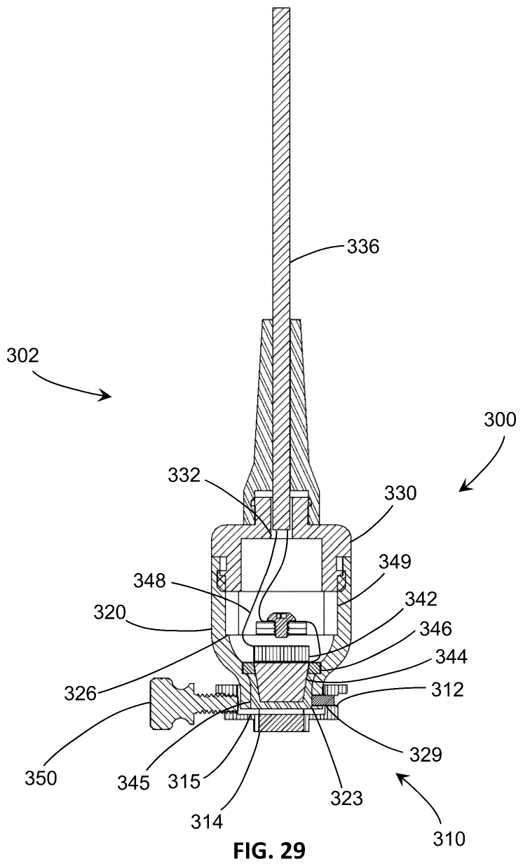

The proximal end of the housing 320 further defines the housing aperture 323, which allows the horn 344 to be in communication with the mounting material and/or implant 10, as shown in FIGS. 20B and 29. As described above, the outer diameter of the proximal end of the housing 320 is less than the inner diameter of the wall 316 of the base 310 such that the proximal end of the housing 320 can insert into the base 310. The base ledge 315 may receive and contact the proximal end of the housing 320 upon its insertion into the base 310. Opposite the housing aperture 323, a housing cap 330 is affixed to the distal end of the housing 320, enclosing, without limitation, the horn 344, horn alignment member 346, transducer 342, transducer flexible circuit 348, and ground 349 within the housing 320. Preferably, the housing cap 330 is designed to maintain axial position and load on the transducer 342 and other components within the housing 320. The interior of the housing 320 may be accessed by selectively engaging or disengaging the housing cap 330. The housing cap 330 is preferably fabricated from the same material as the housing 320, but may be any thermally stable, non-porous material allowing for use of medical and laboratory sterilization methods, including metals such as titanium, aluminum, or stainless steel, other robust materials such as engineering polymers, or any other material which is capable of encapsulating the various components of the device 300, allowing for surface cleaning, and preventing soil and fluid ingress. The housing cap 330 may attach to the housing 320 via compression, threaded engagement, snap-fit, hinges, press fit, clips, adhesive, or any other suitable mechanical connection method. In an alternate embodiment, the housing 320 structure may entirely enclose the transducer 342 and other components without need for a housing cap 330.

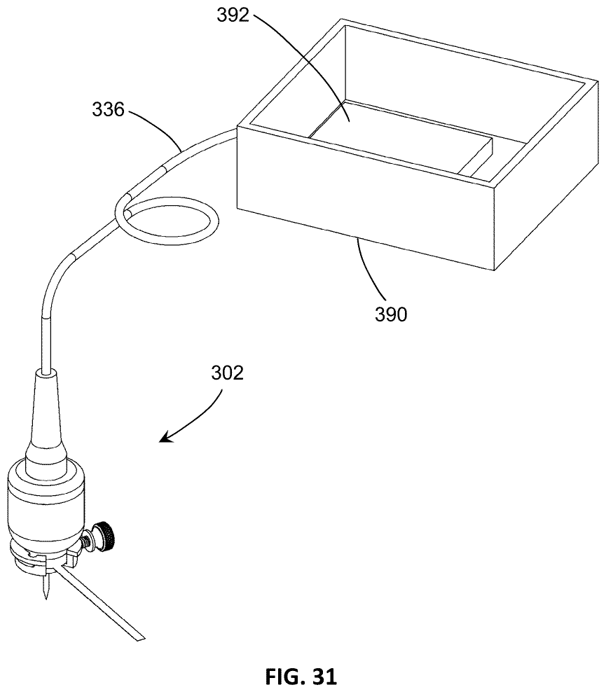

Two elements of this third embodiment work in tandem to transmit power to the assembly 302 and provide acoustic stimulation to the target site 7: the governing assembly and production assembly. The governing assembly transmits power to the production assembly, which produces and delivers acoustic stimulation to the target site 7. The governing assembly includes all components which create and modify the electrical signal delivered to the production assembly. In a preferred embodiment, all elements of the governing assembly, excluding the coaxial power cable 336 and transducer-contacting electrodes 348, will be housed within a control unit 390, preferably a single rack mount enclosure apart from the assembly 302, shown in FIG. 31. The components of the governing assembly include, without limitation: a programmable function generator, a constant gain power amplifier, an adjustable gain signal attenuator, an electrical impedance matching circuit 392, a graphical display (which may preferably be touchscreen-enabled), user interface, the cabling 336 and contact electrodes 348 that contact the transducer 342 within the production assembly, and the power supplies required for each. The production assembly includes all components which create, modify, direct, the mechanical acoustic stimulation delivered to the subject. The components of the production assembly include, without limitation: the transducer 342; the acoustic horn 344 which contacts the transducer 342; a compressive biocompatible polymer 345 which serves as a matching contiguous layer between the subject, the horn 344, and the housing 320; and optionally a horn alignment member 346.

The transducer 342 of the production assembly is capable of generating acoustic vibrations when activated. The terms “transducer,” piezoelectric element,” and “piezo” may be used interchangeably herein to refer to a device generating acoustic vibrations when activated. In embodiments targeting a neural implant 10 or other invasive injury, the transducer 342 is preferably constructed of a single piezo element of a diameter in the range of 2 mm-5 cm, preferably 2 mm-2 cm, more preferably 5 mm-12 mm and thickness in the range of 100 μm-10 mm, preferably 2 mm-5 mm, based on desired targeted tissue 5 area and stimulation frequency. When targeting other injury areas, such as for non-invasive injuries, the transducer 342 is preferably constructed of a single piezo element of a diameter in the range of 5-20 cm, preferably 8-12 cm, and thickness in the range of 100 μm-10 mm, preferably 2 mm-5 mm, based on at least one of the site of injury, entire injured tissue volume or area, desired target site 7 volume and/or area, and stimulation frequency. In further embodiments, the transduce 342 may be any transducer with geometries, size and operative parameters suitable for generating and transmitting acoustic stimulation to a target site 7, including the disc transducer 142 and annular transducer 242, each as fully described herein. The transducer 342 used in the device 300 described herein may preferably produce acoustic vibrations of frequencies in the range of 200 kHz to 5 MHz, preferably 500 kHZ-3 MHz, more preferably 0.5 MHz-2.2 MHz, even more preferably 0.9 MHZ-1.2 MHz and, in one exemplary embodiment, 1.13 MHz. However, transducers 142, 242 may be used with a range of potential frequencies including up to 2 MHz, up to 5 MHz or values in the tens of megahertz, specifically in the range of 5 MHz and 20 MHz. The pressure amplitude of the acoustic waves generated by the transducer may be in the range of 0.1 MPa to 1.5 MPa. The transducer 342 delivers a single sub-threshold, low-intensity ultrasound field having a spatial peak temporal average intensity (I_{SPTA}) in the range of 0.01 W/cm²-5 W/cm², preferably 0.05 W/cm²-2.5 W/cm², more preferably 0.1 W/cm²-2.2 W/cm², even more preferably 0.3-0.5 W/cm². Transducer 342 voltage may be in the range of 50 V and 600 V, preferably 50 V-150 V, or, in one exemplary embodiment, 125 V. Duty cycle percentage in the range of 0.5% and 20%, preferably in the range of 2% and 10%, but more preferably near 5%, and, in one exemplary embodiment, 4%. Pulses, which may also be referred to as bursts, generated by the transducer 342 refer to the intermittent activation of the transducer 342 and associated production of acoustic stimulation during a treatment session. Pulses generated by the transducer 342 may have durations in the range of 1 μs-500 ms, preferably 5 ms-200 ms, more preferably having 20 ms durations, with pulse frequencies in the range of 1 Hz-1 kHz to modulate the transducer 342. An activation period consists of a time period during which pulses are emitted in successive bursts from the transducer and, in exemplary embodiments, are on the order of minutes. In an exemplary treatment session, the transducer is activated for 5 minutes, then off for a rest period of 5 minutes, then activated for 5 minutes for a total treatment session time of 15 minutes. Activation and rest periods during a treatment session may be in the range of 1 minute-15 minutes. During treatment, the transducer 342 may reach a maximum temperature of 27.6° C., but may ideally run at temperatures below 38.5° C., preferably below 38° C., to avoid tissue damage.

Resonance frequency of the transducer 342 varies as the transducer 342 thickness varies. Where the transducer 342 operates in thickness mode, d₃₃, the resonance frequency, fₚₗₐₜₑ, of ultrasound produced may be defined by the equation:

$${f}_{plate} = \frac{1}{2 t} \sqrt{\frac{{C}_{33}^{D}}{ρ}} \qquad ( 4 )$$

where t is the thickness of the piezoelectric element 342, ρ is density, and C₃₃^{D} is relevant elastic stiffness. The applied oscillating electric field, which may be at or near resonance, and poling direction are both through the thickness direction. As an example, for lead zirconate titanate (PZT) piezo ceramics, the approximate thickness to achieve a 1 MHz resonance is 2.1 mm.

As shown in FIGS. 20B and 29, the base aperture 314 which frames the target site 7 of acoustic stimulation, is in communication with the transducer 342 via the horn 344, which is attached to the transducer 342 and directs such stimulation to the target site 7. In a preferred embodiment, the horn 344 has a diameter of ¼ inch at its proximal end and a diameter matching that of the transducer 342 at its distal end. This diameter tapers as the horn 344 approaches the proximal end of the device 300 at an 18-degree angle relative to the longitudinal axis 308. The horn 344 is constructed from an acoustic impedance matched material. The horn 344 can be made from any material suitable for transmitting ultrasonic energy such as steel, stainless steel, aluminum, titanium, magnesium and related alloys. In a preferred embodiment, magnesium is used. Particularly, in a preferred embodiment, a solid, conical horn 344 is machined out of acoustic impedance matched magnesium alloy (AZ31B) allowing for efficient transmission of acoustic energy through cortical bone with low driving voltage with minimal heat production. Magnesium was selected as the preferred horn 344 material following acoustic impedance modeling of the proposed ultrasound transmission pathway between the transducer 342, particularly a PZT-5A piezoceramic transducer 342 (acoustic impedance ˜3,200,000 kg/(m²*s)) and the cancellous bone of the skull (acoustic impedance ˜7,000,000 kg/(m²*s)). AZ31B magnesium alloy has a longitudinal speed of sound and acoustic impedance (10,126,800 kg/(m²*s)) approximating the geometric mean of the PZT-5A piezoceramic transducer and skull for optimal acoustic transmission. AZ31B magnesium alloy was also chosen for its relative high speed of sound (5,770 m/s), reducing transducer 342 nearfield distances allowing for fabrication of small form-factor, high acoustic transmission efficiency quarter wavelength optimized horn 344 lengths within the given operating frequency range. The near field length may be defined by the equation:

$$N = \frac{{D}^{2} f}{4 c} \qquad ( 5 )$$

where N is the near field length, D is the diameter of the transducer 342, f is the operating frequency, and c is the speed of sound within the transmission medium. As an example, with 1 MHz output, an 8 mm diameter transducer 342, and a AZ31B magnesium alloy horn, the near field ends at a length of ˜2.77 mm. Accordingly, the dimensions of the horn 344 are machined to a length minima sufficient to eliminate near field effects of the ultrasound field, a maximal length of 1.25× the wavelength (λ) sufficient to minimize destructive interference at the tissue 5 interface, and a diameter to match the size of the base 310.

The horn 344 may be held within the housing 320 by an alignment member 346. As shown in FIGS. 20A-20B and 27-29, the alignment member 346 may be an annular ring with an inner aperture sized to contain the tapered horn 344 at its distal end. The outer circumference of the alignment member 346 extends radially outward to contact the housing 320, sitting on an alignment member ledge 324 formed within the housing 320, suspending the horn 344 from the housing 320. The alignment member 346 and alignment member ledge 324 together properly align the horn 344 axially and vertically within the housing 320, prevent radial movement of the horn 344 within the housing 320, and center the proximal end of the horn 344 within the center of the housing aperture 323. The horn 344 is secured by the alignment member 346 without contacting the transducer 342 itself. The alignment member 346 induces a radial compression on the horn 342 orthogonal to the axis of ultrasound field propagation (the longitudinal axis 308), minimizing field attenuation from the addition of the component. The alignment member 346 is made from a material that is lower in acoustic impedance than the horn 344 and the housing 320, meaning less acoustic energy is transferred out of the horn 344 through the alignment member 346. In a preferred embodiment, the alignment member 346 is made from Delrin® acetal homopolymer (Wilmington, DE), but may be any suitable material having the foregoing properties, including a polymer material such as nylon polycarbonate, or other similar materials.

In a preferred embodiment, the horn 344 is at least partially encapsulated in a horn compressible biocompatible polymer 345, which, as shown in FIG. 29, extends below the terminal end of the horn 344 opposite the transducer 342, to prevent horn 344 oxidation and corrosion and to maintain the integrity of the transducer 342. At a minimum, the polymer 345 contacts both the terminal end of the horn and the mounting material or target surface 7, as applicable. Acoustic energy from the transducer 342 is faithfully transmitted through the polymer 345, serving as a deformable or compressible acoustic matching layer between the horn 344 and the subject. The polymer 345 substantially surrounds the horn 344 and fills space between the inner surface of the housing 320, the horn 344, and the horn alignment member 346. The horn alignment member 346 and transducer 342 are in direct contact with the horn 344 without polymer 345 therebetween. In a preferred embodiment, the thickness of this polymer 345 is approximately 0.04 inches and the portion of the polymer 345 closest to the proximal end of the assembly 302 is preferably coextensive with the housing aperture 323, but may be within 0.01 inches of the aperture 323 to account for manufacturing and measuring tolerances.

The polymer 345 material should be sufficiently flexible or soft to conform to the targeted biologic surface geometry of the subject, be biocompatible so as to avoid initiating an immune response in the subject, be stable such that it not decay from use, and be slightly compressible with little to no change in acoustic performance. Particularly, the polymer 345 material should exhibit consistent acoustic transmission characteristics across a range of induced pressures in the therapeutic frequency ranges and throughout the other treatment parameters disclosed herein. As the housing 320 is lowered onto the base 310, this biocompatible polymer 345 may necessarily slightly compress to conform to the mounting material within the base aperture 314. At fullest compression of the polymer 345, that is, when the housing 320 is fully lowered into contact with the mounting material within the base aperture 314, there should be substantially no attenuation in transmission, allowing acoustic stimulation to faithfully propagate from the transducer 342, through the horn 344, to the target tissue 7. The alignment tab 329 may be positioned anywhere along channel 312 based on the mounting material thickness. However, the polymer 345 should be transmissive with the alignment tab 329 at any point of the channel 312, that is, the material should still be transmissive of acoustic vibrations without compression. The biocompatible polymer 345 should meet defined biological safety thresholds as set forth in the ISO 10993 set of standards. for user safety and regulatory compliance. The biocompatible polymer 345 can be made from material in the silicone family, rubber family, thermoplastic elastomer family, or any other suitable flexible, compressible material. In a preferred embodiment this material is a silicone having the foregoing traits. One example of a material that meets all these requirements is silicone NuSil MED4-4220 made by Avantor Inc. (Radnor, PA). Following curing, the foregoing compressible biocompatible polymer 345 is a soft 17A durometer with a tensile strength of 660 psi, and an elongation of 580%.

In a preferred embodiment, as described above, the electrical impedance matching circuit 392 is located within the control unit 390, separate from the assembly 302. The electrical impedance matching circuit 392 is designed to receive high frequency driving voltages in the range of 1 V and 1,000 V for signal conditioning prior to driving the transducer 342. The electrical impedance matching circuit 392 is designed for matching the high electrical impedance of the transducer 342, which may be in the range of 10Ω-10,000Ω, preferably ˜300Ω-500Ω, to the low impedance driving electronics of the governing assembly, approximately 50Ω, improving electrical efficiency and lowering overall power requirements.

In alternate embodiments, the electrical impedance matching circuit 392 may be located within the housing 320 and directly connected to the transducer 342 via conductive or non-conductive epoxy or other suitable methods of electrical connection. When present, a carrier holds the electrical impedance matching circuit 392 within the housing 320 apart from the transducer 342, the carrier being suspended and resting on a carrier ledge formed from the housing 320.

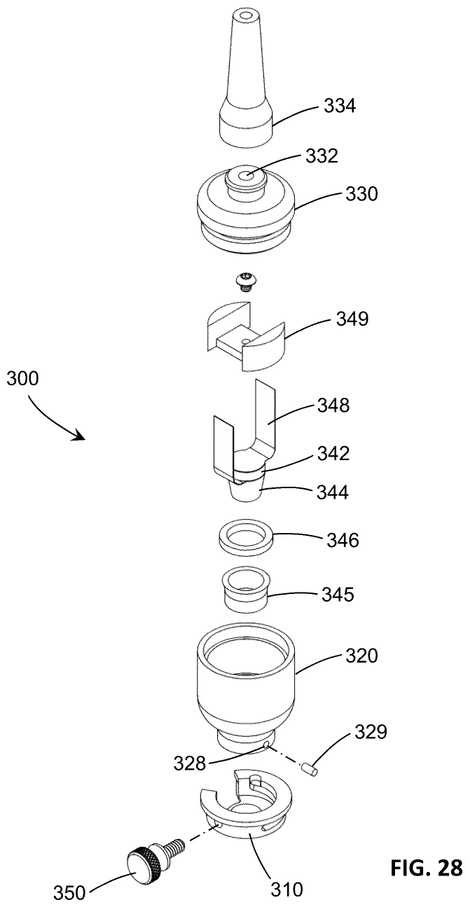

When the electrical impedance matching circuit 392 is located within the control unit 390, a flexible circuit 348 provides the connection between the transducer 342 and the governing assembly, as shown most clearly in FIGS. 28-29. This flexible circuit 348 serves as the positive and negative electrical contacts required for power delivery to the transducer 342. In a preferred embodiment, the flexible circuit 348 consists of flexible copper traces attached to the poled surfaces of the transducer 342, which provides a large connection surface area more resistant to mechanical stress and suitable for providing even excitation of the transducer 342. Silver doped conductive epoxy attaches the flexible copper traces 348 to the transducer 342, however, it is feasible to use non-conductive epoxy, particularly Loctite M-31 CL (Henkel Corporation, Düsseldorf, Germany), if the epoxy layer is thin enough to mate the two electrically conductive surfaces, generally being several microns at most. In alternate embodiments, electrical coupling was performed through soldering of wires 348 directly to the face of the transducer 342 contacts. This high temperature connection method results in non-homogenous excitation of the transducer 342 and risks de-poling and degradation of transducer 342 performance.

A power cable 336 transmits drive voltage from the governing assembly to the transducer 342, extending from the control unit 390 to the housing 320 through an aperture 332 formed in the housing cap 330 to arrive at the transducer 342, shown in FIG. 29. A cable strain relief 334, shown in FIGS. 27-29, may be provided at the aperture 332, attaching externally to the housing 320 to maintain the integrity of the power cable 336.

Additionally, the housing 320 may include a ground element 349 which is attached to the transducer 342, in a preferred embodiment, by a screw. The ground element 349 is present to keep the subject safe from shock, fire, short circuit, or other electrical hazards. This ground element 349 sits within the distal end of the housing 320, above the transducer 342, on a ground ledge 326 formed from the housing 320, shown in FIG. 29.

Various materials may be utilized throughout the assembly, as shown in Table 1, materials of various hardness and acoustic impedance may be used for different applications. The acoustic impedance of these materials influences how ultrasound will propagate through each individual element as well as between elements and their relative hardness. The softer of these materials can serve as a couplant to provide a tight, conformable interface between layers of the assembly 302 and aid in energy transfer. The harder of these materials can serve as a fixation or structural component of the assembly 302 to keep other components in alignment or provide rigidity. Certain materials can have a wide range of impedance and hardness based on their composition, for example, the impedance and hardness of the tungsten-doped epoxy may change based on the ratio of tungsten to epoxy.

TABLE 1

| | | Acoustic | |
|---|---|---|---|
| Material | Use in assembly | impedance | Hardness |
| Lead Zirconate Titanate | Transducer | 30-35 | Very hard |
| (PZT) | | | |
| Polyvinyl alcohol (PVA) | Horn/couplant | 1-3 | Soft |
| Tungsten-doped epoxy | Horn/couplant | 1-40 | Variable |
| Magnesium | Horn | 10 | Hard |
| Aluminum | Horn | 17.3 | Hard |
| Delrin acetal homopolymer | Horn alignment | 3.45 | Hard |
| | member | | |
| Silicone | Horn/compressible | 1-2 | Soft |
| | polymer material/ | | |
| | couplant | | |
| Dental acrylic (PMMA) | Mounting material | 3.26 | Hard |
| | (Biological | | |
| | matching layer) | | |
| Hard biological tissue | Biological contact | 3-7.5 | Hard |
| (bone) | | | |
| Soft biological tissue | Biological contact | 1.5-1.6 | Soft |
| (skin) | | | |
| Water | | 1.48 | Soft |

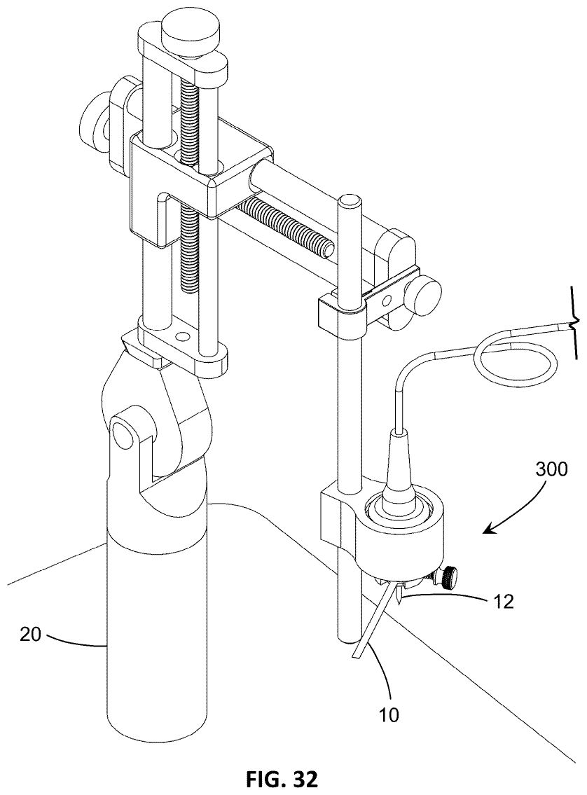

In some embodiments, the device 300 may be mounted to a stereotaxic frame 20 when in use, as shown illustratively in FIG. 32. The frame 20 may hold the device 300 in proximity to the subject and target site 7, or the device 300 may be independently mounted to the subject. In some embodiments, the frame 20 may attach to the device 300 via an adapter configured to hold the device 300 by either wrapping around the device 300 or by being inserted between the pieces of the assembly 302 itself, such as by attaching to one or more features of the base 310 or housing 320. In alternate embodiments, the frame 20 may attach by screw, clamp, adhesive, press-fit, or any other similar method.

The control unit 390, shown in FIG. 31, has the ability to adjust parameters such as acoustic stimulation frequency and amplitude which are integrated into the user interface via an API or GUI, where the user has the ability to modulate each parameter higher or lower via digital or analog controls. This could be performed to either determine optimal parameters for therapy or to evaluate different physiological effects from a user-defined parameter space. It is expected that such parameters would be fixed for the duration of a treatment session and changed in between sessions, though there may be instances where parameters could be changed during a session, such as to determine if any immediate effects are seen with changes in acoustic output. Further, an automatic timer could be integrated into the control unit 390 to shut off the excitation signal to the transducer 342 to end stimulation at desired time. Alternatively, a closed loop system with an adjunct sensor could be implemented where a desired outcome is reached, and the assembly 302 is turned off. Possible desired outcomes in treatment following implantation of a neural implant 10 or other invasive injury include eliciting an increase in localized perfusion or oxygenation around the indwelling neural electrode or inserted body, increased production of neurotrophic factors, or change in neuronal firing. Possible desired outcomes in treatment following a native non-invasive injury include, but are not limited to, stabilizing brain blood barrier, reducing neuro inflammation, and reducing cellular death. Following these outcomes, as applicable, the adjunct sensor may shut off the transducer 342 via the control unit 390. This sensor, for example, may consist of an adjunct bio-electrical circuit that changes resistance with changing levels of biological analytes as they bind to antibodies or ligands on the circuit and a threshold resistance change switches the excitation off or switching of a binary signal (from on to off) when a threshold biological condition, as measured by the indwelling neural electrode 12, is reached.

Some stimulation-only systems aimed at the treatment of implants 10 incorporate constant electrical current sources that increase electrical voltage as the FBR develops and insulates the implant 10 with cells. This is based on the standard Ohm's Law, where an increase in resistance (from cells around the implant 10) is countered by an increase in voltage to maintain current. However, this approach affects the battery life of the implant 10, and may affect local tissue. The present assembly 302 could be used as an alternative to mitigate this issue and increase battery life of the implant 10.

Treatment protocols, being the number of times each day or week that treatment sessions occur, and the parameters of wave cycles, pulse duration, pulse duration repetition rate, and timing of activation and rest periods for each treatment session, could be tailored for individual subjects or for various injuries. Activation periods are the time of the treatment session during which acoustic stimulation pulses are delivered to a subject. The parameters of wave cycles and pulse durations consist of the amplitude or intensity, duty cycle, and pulse length delivered by the assembly 302. The tailored treatment protocol could be based on aspects such as but not exclusive to the age, health history, nature of the injury, exact anatomical placement, or healing aspects of the subjects, or, if applicable, the number of electrodes 12 or drive characteristics of the implant 10.

The present assembly 302 may be operated according to any of the parameters or methods of use described above with respect to other devices 100, 200 and their respective transducers 142, 342. Any of the parameters and methods described herein may be used to treat injury associated with the insertion of an implant 10 or may be used non-invasively on a subject near the site of a native injury. When operating non-invasively, the device 100, 200 or assembly 302 is placed on a subject proximate to a target site 7 to transmit acoustic stimulation through intervening tissue, such as transcranially. The constituent parts of the devices 100, 200 or assembly 302 may be scaled to encompass a larger target area typically associated with a non-invasive native injury, however, treatment protocols remain similar. Briefly, the operating frequency of the transducer 342 will be in the range of 900 kHz-1.2 MHz. Pulse durations will occur on the scale of 20 ms with a repetition rate of two pulses per second for a 5-minute activation period, in other words the transducer 342 will deliver 20 ms pulses of acoustic stimulation to the subject tissue 7 twice every second for an activation period of 5 minutes. Multiple 5-minute activation periods can be interleaved with rest periods to create a more complex single-day treatment paradigm with changes in daily treatment sessions and activation period frequency also possible.

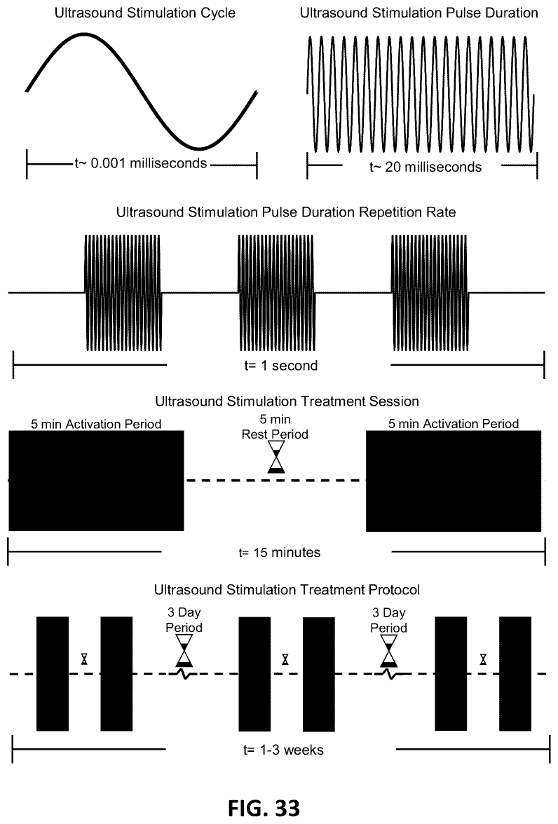

For the treatment methods described herein, each treatment protocol is comprised of a number of treatment sessions, each treatment session is comprised of one or more activation periods and rest periods, each activation period is comprised of a repetition of pulse durations, and each pulse duration is comprised of a number of wave cycles. FIG. 33 shows an exemplary embodiment of the treatment methods described herein. Various parameters of the stimulation and treatment session shown in FIG. 33 may be varied to suit different applications. As shown in FIG. 33, a transducer 342 emits a number of wave cycles which together form a pulse. Wave cycles are emitted on the order of one cycle per approximately 0.001 ms, for the duration of the pulse. Pulses are repeated at a repetition rate of 3 pulse durations per second during an activation period. As shown, a treatment session consists of, first, 3 pulses of ultrasonic stimulation per second being delivered for an activation period of 5 minutes, second, a 5-minute rest period, and, third, 3 pulses of ultrasonic stimulation per second being again delivered for an activation period of 5 minutes. These treatment sessions can be repeated as part of a treatment protocol, as shown, for 1-3 weeks following injury by providing three days between treatment sessions.

In the treatment of both invasive and non-invasive injuries, the ultrasonic field produced by the transducer 342 may be expanded by changing transducer 342 parameters to encompass both the damage locus and surrounding affected tissue, to preferably apply ultrasonic vibrations to the entire injured tissue, which is also the target site 7. The ultrasonic field may be altered by a variety of factors as described herein and by other factors known in the art. The acoustic field is defined by a near field adjacent to the transducer 342 and passing through the affected tissue area, and a far field located past a transition point, penetrating deeper into target tissue 7 to reach both the damage locus and extended affected tissue area.

Since many modifications, variations and changes in detail can be made to the described preferred embodiments, it is intended that all matters in the foregoing description and shown in the accompanying drawings be interpreted as illustrative and not in a limiting sense. Thus, the scope of the invention should be determined by the appended claims and their legal equivalents.

What is claimed is:

1. An assembly for reducing injury response at a target site being at least a portion of injured tissue of a subject, said assembly comprising:
a base having a proximal end, an opposite distal end, a base aperture formed in said proximal end, and a channel formed in and extending along at least a portion of said base between said proximal and distal ends, said base securable to the subject with said base aperture in proximity to the target site;
a housing selectively secured to and variably adjustable relative to said base, said housing having a proximal end, an opposite distal end, a housing aperture formed in said proximal end of said housing and aligned with said base aperture, and an alignment member extending from said housing and slidably received within said channel of said base;
a transducer retained within said housing, said transducer capable of generating acoustic vibrations sufficient to reduce the injury response in the subject at the target site when activated; and
a horn retained within said housing, said horn having a distal end in contact with said transducer and a proximal end terminating at said housing aperture, said horn transmitting said acoustic vibrations from said transducer to said target site.

2. The assembly as recited in claim 1, wherein said housing is variably adjustable relative to said base along a longitudinal axis defined substantially perpendicular to said base aperture.

3. The assembly as recited in claim 2, wherein said channel comprises a slope along its length, said alignment member of said housing slidingly moveable along said channel to adjust said housing along said longitudinal axis relative to said base.

4. The assembly as recited in claim 3, said housing is selectively rotatable about said longitudinal axis to move said alignment member along said channel and adjust said housing along said longitudinal axis relative to said base.

5. The assembly as recited in claim 3, wherein said slope is an angle in the range of 1 to 10 degrees.

6. The assembly as recited in claim 2, said base further comprising a base wall disposed about said longitudinal axis and having a thickness, and wherein said channel is formed in said base wall and either (i) is a portion of said thickness of said base wall, or (ii) extends through the entirety of said thickness of said base wall.

7. The assembly as recited in claim 2, said base further comprising a top surface at said distal end of said base, and an opening defined in said top surface, said opening being continuous with said channel, said opening and channel being sized to receive and slidingly retain said alignment member therein.

8. The assembly as recited in claim 2, said base further comprising a base ledge extending radially outwardly from said base aperture and said longitudinal axis, said base ledge sized and configured to receive and retain said proximal end of said housing.

9. The assembly as recited in claim 1, further comprising a locking mechanism comprising a receiver in said base and a stem correspondingly configured to engage said receiver, said locking mechanism selectively actuated to secure said housing to said base and prevent further movement of said housing with respect to said base.

10. The assembly as recited in claim 9, wherein said channel is continuous and said locking mechanism is selectively actuated to secure said housing relative to said base at any point along said channel.

11. The assembly as recited in claim 9, wherein said locking mechanism is actuated by one of (i) friction between said stem and said receiver; (ii) corresponding threading between said stem and said receiver; (iii) biasing of said stem against said housing; and (iv) biasing of said stem against said base, wherein said stem is a part of said housing.

12. The assembly as recited in claim 1, further comprising a polymer disposed contiguously between said proximal end of said horn and a surface of the subject at said base aperture when mounted thereon, said polymer transmitting said acoustic vibrations and being compressible with variable adjustment of said housing relative to said base without substantially altering properties of said acoustic vibrations transmitted therethrough.

13. The assembly as recited in claim 12, wherein said polymer is biocompatible and is one of silicone, rubber, and thermoplastic elastomer.

14. The assembly as recited in claim 13, wherein said polymer comprises a durometer of 17A, a tensile strength of 660 psi and an elongation of 580%.

15. A method of reducing injury response at a target site of tissue of a subject, said method comprising:
positioning the device of claim 1 in proximity to the target site, the target site being at least a portion of injured tissue;
generating acoustic vibrations by activating said transducer for at least one activation period;
transmitting said acoustic vibrations to the target site; and
applying said acoustic vibrations to the target site sufficient to reduce injury response in the subject at the target site.

16. The method as recited in claim 15, wherein the injured tissue includes at least one of: (a) a damage locus being one of: (i) an insertion site of a foreign body, and (ii) a site of a non-invasive injury; and (b) surrounding affected tissue proximate to a damage locus.

17. The method as recited in claim 16, wherein the injured tissue comprises one of:
(i) an insertion site of one of an electrode and an implant, and the surrounding affected tissue undergoing a foreign body response; and
(ii) a site of non-invasive injury and the surrounding affected tissue undergoing microgliosis.

18. The method as recited in claim 16, wherein said non-invasive injury is one of stroke, epilepsy, percussive force, ischemia, aneurysm, hemorrhage, encephalitis, and traumatic brain injury.

19. The method as recited in claim 15, wherein applying said acoustic vibrations to the target site includes applying at least one of a frequency and intensity of vibrations sufficient to stimulate release of at least one endogenous neurotrophic factor in the injured tissue.

20. The method as recited in claim 15, wherein positioning the device includes positioning the device on the subject in proximity to the target site and spaced apart from the target site by intervening tissue, and wherein transmitting said acoustic vibrations includes transmitting said acoustic vibrations through the intervening tissue.

21. The method as recited in claim 15, wherein said acoustic vibrations are in the ultrasonic frequency range.

22. The method as recited in claim 15, wherein activating said transducer comprises at least one of:
(i) activating said transducer at a voltage being one of: in the range of about 50 to 600 V, in the range of about 50 to 150 V, and of about 125 V;
(ii) activating said transducer at a duty cycle percentage being one of: in the range of about 0.5% to 20%, in the range of about 2% to 10%, of about 5%, and of about 4%;
(iii) activating said transducer sufficient to produce said acoustic vibrations having a frequency of one of: in the range of about 200 kHz to 20 MHz, in the range of about 200 kHz to 5 MHz, in the range of about 500 KHZ to 3 MHz, in the range of about 0.5 MHz to 2.2 MHz, in the range of about 0.9 MHz to 1.2 MHZ, and about 1.13 MHz;
(iv) activating said transducer sufficient to produce said acoustic vibrations having a pulse duration of one of: in the range of about 1 μs-500 milliseconds, in the range of about 5 milliseconds to 200 milliseconds, and about 20 milliseconds; and
(v) activating said transducer sufficient to produce said acoustic vibrations having a spatial peak temporal average intensity of one of: in the range of about 0.01 W/cm² to 5 W/cm², in the range of about 0.05 W/cm² to 2.5 W/cm², in the range of about 0.1 W/cm² to 2.2 W/cm², and in the range of about 0.3 W/cm² to 0.5 W/cm².

23. The method as recited in claim 15, further comprising repeating steps (b) through (d) for a treatment session, said treatment session comprising one of:
(i) turning said transducer on for said activation period in the range about 1 to 15 minutes, turning said transducer off for a rest period in the range about 1 to 15 minutes, and repeating from 2 to 10 times; and
(ii) turning said transducer on for said activation period of 5 minutes, turning said transducer off for a rest period of 5 minutes, and turning said transducer on for said activation period of 5 minutes, for a total treatment time of 15 minutes.

24. The method as recited in claim 23, further comprising performing said treatment session at one of: once every day during the week following initial injury to the target tissue, once every other day during the second week following initial injury to the target tissue, and once every three days during the second week following initial injury to the target tissue.

25. The method as recited in claim 15, wherein applying said acoustic vibrations comprises creating an acoustic field of said acoustic vibrations at the target site, said acoustic field surrounding substantially an entirety of the injured tissue.

26. The method as recited in claim 25, wherein said acoustic field comprises a near field and a far field separated by a transition point, said far field having a wider diameter than said near field; and at least one of (i) said near field and (ii) said far field surrounding substantially an entirety of the injured tissue.

27. The method as recited in claim 25, further comprising modulating said acoustic field by changing one of: (i) a frequency of said acoustic vibrations, and (ii) a diameter of said transducer.

28. The method as recited in claim 15, wherein applying said acoustic vibrations comprises creating an acoustic field of said acoustic vibrations at the target site; and further comprising creating overlapping acoustic fields of said acoustic vibrations at the target site, at least one of (i) the overlapping portion of said acoustic fields and (ii) a space between said overlapping acoustic fields surrounding at least a portion of the injured tissue.
